(19) United States
(12) Patent Application Publication
(10) Pub. No.: US 2022/0023666 A1
(43) Pub. Date: Jan. 27, 2022

(54) ARTIFICIAL NEURAL NETWORK BASED RADIOTHERAPY SAFETY SYSTEM

(71) Applicant: University Health Network, Toronto (CA)

(72) Inventors: Young-Bin Cho, Mississauga (CA); Mohammad Khairul Islam, Oakville (CA); David Anthony Jaffray, Etobicoke (CA); Bernhard Dieter Norrlinger, Mississauga (CA); Makan Farrokhkish, North York (CA); Robert K. Heaton, Toronto (CA)

(21) Appl. No.: 17/311,968

(22) PCT Filed: Dec. 10, 2019
(86) PCT No.: PCT/CA2019/051780

§ 371 Date: Jun. 8, 2021

Related U.S. Application Data
(60) Provisional application No. 62/777,701, filed on Dec. 10, 2018.

(51) Int. Cl. A61N 5/10; G06N 3/04; G16H 20/40; G01T 1/02

(52) U.S. Cl. CPC A61N 5/1071; A61N 5/1045; G01T 1/02; G16H 20/40; G06N 3/0454

(57) ABSTRACT

Various embodiments are described herein of radiation systems and methods for monitoring radiation dose are provided monitoring an amount of radiation in a radiation beam generated by a radiation source for a radiation treatment session, where a radiation sensor is used to provide an actual radiation measurement and an Artificial Neural Network (ANN) engine is used to generate a predicted radiation measurement based on a plurality of feature values for features including radiation field segments from the radiation treatment plan data for the radiation treatment session. The difference between the actual radiation measurement and the predicted radiation measurement is used to determine whether the radiation system is operating in a predetermined safe operation range.

### CROSS-REFERENCE

This application claims the benefit of U.S. Provisional Patent Application No. 62/777,701, filed Dec. 10, 2018, and the entire contents of U.S. Provisional Patent Application No. 62/777,701 is hereby incorporated by reference.

### FIELD

The present disclosure relates to systems and methods for quality assurance in the field of radiation treatment for real-time, off-line, pre-treatment or post-treatment verification of the delivery of radiation dose.

### BACKGROUND

Radiation treatment for cancer has improved significantly with the advent of modern treatment planning and delivery techniques such as Intensity Modulated Radiation Therapy (IMRT) (Webb, 1994), and Volumetric Arc Radiation Therapy (VMAT) (Boyer et al., 1999). Combined with high quality on-line imaging modalities such as cone beam computed tomography (CBCT) (Jaffray et al., 2000) and Magnetic Resonance Imaging (MRI) (Raaijmakers, 2007), precise dose delivery has become feasible by utilizing smaller planning margins with a goal of maintaining the same therapeutic dose to the target while simultaneously minimizing dose to surrounding organs. However, complex treatment plans pose the increasing potential for errors during planning, quality assurance, and dose delivery compared to simpler delivery techniques. Detection of these errors may be even more difficult and may go unnoticed when frequent and on-line (i.e. patient on the treatment couch) plan adjustment is required for adaptive radiotherapy. Although the quality of a treatment plan is validated only once before the start of the course of radiation therapy, using conventional methods and quality assurance equipment, monitoring daily fractional dose is not a practice even at the most advanced health care institutions due to lack of availability of suitable verification system. Monitoring of treatment beams daily, with conventional dose measurement methods, would require additional staff and treatment unit time, which is considered to be impractical. This deficiency has also prevented implementation of daily adaptive radiation therapy, when a treatment plan will be developed or selected based on the on-line (while the patient is on the treatment couch) imaging of patient anatomy.

Several Radiation Quality Check Systems (RQCS) exist that can validate accuracy of radiation energy fluence. An example of one such RQCS is a large area ion-chamber with a spatial gradient that can be positioned between the beam source and the patient for real-time dose monitoring (as further described in WO2008006198). In a RQCS, the treatment beam is monitored and verified by comparing the output of a radiation sensor device used by the RQCS with the predicted signal calculated by an analytic numerical model based on the physics of the beam geometry, treatment unit characteristics, and detector unit characteristics. The development of an analytic calculation model often requires laborious measurements, data preparation, and sophisticated tuning of the model parameters. The performance of the analytic model has been found to be less than satisfactory in highly irregularly shaped beam geometrical situations.

### SUMMARY OF VARIOUS EMBODIMENTS

According to one broad aspect of the teachings herein, there is provided a radiation dose monitoring system for monitoring an amount of radiation in a radiation beam generated by a radiation source for a radiation treatment session, wherein the system comprises a radiation sensor that is positioned in a path of the radiation beam and is configured to provide an actual radiation measurement of an amount of radiation in the radiation beam; an interface unit, operatively coupled to the at least one radiation sensor; a memory unit; and a processor, operatively coupled to the interface unit and the memory unit, the processor being configured to: obtain radiation treatment plan data for the radiation treatment session; extract a plurality of feature values for features of radiation field segments from the radiation treatment plan data for the radiation treatment session; generate a predicted radiation measurement using an artificial neural network engine that receives the plurality of feature values as inputs; and determine an error measurement between the actual radiation measurement.

In at least one embodiment, the artificial neural network engine is configured to generate predicted radiation measurements in real-time, off-line, pre-treatment or post-treatment quality assurance.

In at least one embodiment, the processor is further configured to send a notification output signal to an operator of the radiation source when the error measurement is outside a predetermined safe operation range for the amount of radiation defined in the radiation treatment plan data.

In at least one embodiment, the processor is further configured to generate a control signal that is provided to the radiation source to stop the generation of the radiation beam when the error measurement is outside of a predetermined safe operating range for the amount of radiation defined in the radiation treatment plan data.

In at least one embodiment, the processor is further configured to generate a control signal that is provided to the radiation source to adjust the amount of radiation in the radiation beam that is generated by the radiation source when the error measurement is outside of a predetermined safe operating range for the amount of radiation defined in the radiation treatment plan data.

In at least one embodiment, the features of the radiation field segments comprise spatial variation of energy fluence, positional sensitivity of the radiation sensor, contribution of a secondary radiation source and shape of field opening area.

In at least one embodiment, the radiation sensor comprises a large area gradient ion chamber and the ANN engine is optionally configured to use 10 features of the radiation field segments as input features.

In at least one embodiment, the radiation sensor comprises two large area gradient ion chambers in a stacked configuration having parallel and opposing gradients or having orthogonal gradients, each ion chamber being adapted to provide an output vale for the actual radiation measurement, and the ANN engine is optionally configured to use 10 features of the radiation field segments as input features.

In at least one embodiment, the features for the variation of energy fluence include: ƒ₄=∫ΨₚrdA and ƒ₅=∫Ψₚr²dA where Ψₚ is energy fluence due to a primary radiation source, r is a radial distance from a center of a treatment beam area defined by jaw and Multileaf Collimator geometry of the radiation source and the integral is taken over the treatment beam area.

In at least one embodiment, the features for the positional sensitivity of the radiation sensor include: ƒ₁=∫ΨₚdA, ƒ₂=∫ΨₚxdA and ƒ₃=∫Ψₚx²dA where Ψₚ is energy fluence due to a primary radiation source, x is a direction of a Multileaf Collimator (MLC) or a direction of detector sensitivity and the integral is taken over the treatment beam area defined by jaw and MLC geometry of the radiation source.

In at least one embodiment, the feature of contribution of a secondary radiation source include ƒ₆=∫ΨₛdA where Ψₛ is energy fluence due to a secondary radiation source, and the integral is taken over the treatment beam area defined by jaw and MLC geometry of the radiation source.

In at least one embodiment, the feature of contribution of shape of field opening area include f₇=f₁/(f₁+ε₁*f₆) and f₈=f₆/(f₁+ε₂*f₆) where 0<ε₁<1 and 0<ε₂<1 and ε₁ does not have to be equal to ε₂.

In at least one embodiment, the features of the shape of field opening area include ƒ₉=A_{MLC}/R_{MLC} and ƒ₁₀=A_{MLC}/A_{Jaw} where A_{MLC} and A_{Jaw} are opening areas of an MLC and Jaws of the radiation source, respectively, and R_{MLC} is a rectangular area defined by a maximum separation of an MLC pair in the radiation field.

In at least one embodiment, the radiation sensor comprises a plurality of point detectors in a two dimensional array with Y rows and N columns where each point detector provides an output value for the actual radiation measurement and the ANN engine employs an ANN for each of the point detector or a single ANN with F*Y*N inputs to generate a two dimensional array of output values for the predicted radiation measurement, where F is a number of input features and F, Y and N are integers greater than zero.

In at least one embodiment, the features for the variation of energy fluence include: ƒ₄=∫ΨₚrdA and ƒ₅=∫Ψₚr²dA where Ψₚ is energy fluence due to a primary radiation source, r is a radial distance from a radiation detector center and the integral is taken over an area around each of the point detectors.

In at least one embodiment, the features of the primary fluence measured by the radiation sensor include: ƒ₁=∫ΨₚdA, ƒ₂=∫Ψₚ*G(s)dA and ƒ₃=∫Ψₚ*G(l)dA where Ψₚ is energy fluence due to a primary radiation source, and G(s) and G(l) are small and large Gaussian kernels and the integral is taken over an area around each of the point detectors.

In at least one embodiment, the feature of contribution of a secondary radiation source include ƒ₆=∫ΨₛdA, ƒ₇=∫Ψₛ*G(s)dA and ƒ₈=∫Ψₛ*G(l)dA, where Ψₛ is energy fluence due to a secondary radiation source, G(s) and G(l) are small and large Gaussian kernels and the integral is taken over an area around each of the point detectors.

In at least one embodiment, the feature for accounting for edges of the radiation beam segments includes ƒ₉=∫Ψₚ*E(s)dA where E(s) is an edge filter and the integral is taken over an area around each of the point detectors.

In at least one embodiment, the radiation sensor comprises Y line detectors that each provide an output value for the actual radiation measurement and the ANN engine employs an ANN for each line detector or a single ANN with F*Y inputs to generate a linear array of output values for the predicted radiation measurement, where F is a number of input features and F and Y are integers greater than zero.

In at least one embodiment, the radiation sensor comprises a 3D arrangement of radiation detectors, where the 3D arrangement includes N groups of Z radiation detectors and the ANN engine employs an ANN for each group or a single ANN with N*Z*F inputs and N*Z outputs, where F is an integer representing the number of input features that are used where F, N and Z are integers that are greater than zero.

In at least one embodiment, the ANN engine is configured to use additional input features including at least one of radiation source model, MLC model, beam energy, type of radiation sensor, and radiation sensor location.

In at least one embodiment, the ANN engine is configured to use additional input features comprising patient geometry at a treatment region, location of the patient on a treatment table and radiation sensor location including immediately positioned before the patient for entrance beam monitoring or positioned after the patient for exit beam monitoring.

In at least one embodiment, the ANN engine is configured to use a multi-layer perceptron (MLP) neural network or a convolutional neural network.

In at least one embodiment, the ANN is the MLP neural network and comprises an input layer having a plurality of input nodes equal to the number of features, at least one hidden layer with a plurality of hidden nodes and an output layer with an output node.

In at least one embodiment, the nodes of the multi-layer perceptron neural network are adapted to use a sigmoidal function as a weight factor.

In at least one embodiment, the MLP neural network comprises one hidden layer.

In at least one embodiment, the ANN is trained using radiation treatment parameters for a variety of Quality Assurance (QA) and Area Output Factor (AOF) fields, and training data including data that was obtained from various types of radiation source manufacturers, different radiation source models including different collimator types, different amounts of beam energy, and different beam calibration units.

In at least one embodiment, the ANN engine is configured to use N ANNs to generate N intermediate predicted radiation measurements that are statistically combined to provide the predicted radiation measurement, where N is an integer greater than one.

In at least one embodiment, the ANN engine is configured to use an ANN that has been trained using training set data obtained for treating the same treatment region that is being treated in the radiation treatment session.

In at least one embodiment, the ANN engine is configured to use N ANNs to generate N intermediate predicted radiation measurements that are statistically combined to provide the predicted radiation measurement, where N is an integer greater than one where each ANN has been trained using training set data obtained for treating the same treatment region that is being treated in the radiation treatment session.

In another broad aspect, in accordance with the teachings herein, there is provided a method for monitoring an amount of radiation in a radiation beam generated by a radiation source for a radiation treatment session, wherein the method comprises: obtaining an actual radiation measurement of an amount of radiation in the radiation beam from a radiation sensor that is positioned in a path of the radiation beam; and at a processor: extracting a plurality of feature values for features of radiation field segments from the radiation treatment plan data for the radiation treatment session; generating a predicted radiation measurement using an artificial neural network engine that receives the plurality of feature values as inputs; and determining an error measurement between the actual radiation measurement and the predicted radiation measurement.

In at least one embodiment, the artificial neural network engine is configured to generate predicted radiation measurements in real-time, off-line, pre-treatment or post-treatment quality assurance.

In at least one embodiment, the method further comprises sending a notification output signal to an operator of the radiation source when the error measurement is outside a predetermined safe operation range for the amount of radiation defined in the radiation treatment plan data.

In at least one embodiment, the method comprises generating a control signal that is provided to the radiation source to stop the generation of the radiation beam when the error measurement is outside of a predetermined safe operating range for the amount of radiation defined in the radiation treatment plan data.

In at least one embodiment, the method comprises generating a control signal that is provided to the radiation source to adjust the amount of radiation in the radiation beam that is generated by the radiation source when the error measurement is outside of a predetermined safe operating range for the amount of radiation defined in the radiation treatment plan data.

In at least one embodiment, the radiation sensor comprises a large area gradient ion chamber, and the method comprises optionally configuring the ANN engine to use 10 features of the radiation field segments as input features.

In at least one embodiment, the radiation sensor comprises two large area gradient ion chambers in a stacked configuration having parallel and opposing gradients or having orthogonal gradients, each ion chamber being adapted to provide an output vale for the actual radiation measurement, and the method optionally comprising configuring the ANN engine to use 10 features of the radiation field segments as input features.

In at least one embodiment, the method comprises configuring the ANN engine to use additional input features including at least one of radiation source model, MLC model, beam energy, type of radiation sensor, and radiation sensor location.

In at least one embodiment, the method comprises configuring the ANN engine to use additional input features comprising patient geometry at a treatment region, location of the patient on a treatment table and radiation sensor location including immediately positioned before the patient for entrance beam monitoring or positioned after the patient for exit beam monitoring.

In at least one embodiment, the method comprises using N ANNs to generate N intermediate predicted radiation measurements that are statistically combined to provide the predicted radiation measurement, where N is an integer greater than one.

In at least one embodiment, the method comprises employing an ANN that has been trained using training set data obtained for treating the same treatment region that is being treated in the radiation treatment session.

In at least one embodiment, the method comprises employing N ANNs to generate N intermediate predicted radiation measurements that are statistically combined to provide the predicted radiation measurement, where N is an integer greater than one where each ANN has been trained using training set data obtained for treating the same treatment region that is being treated in the radiation treatment session.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A is a schematic diagram of an example embodiment of a radiation dose monitoring system.

FIG. 1B is a schematic diagram of an example embodiment of a Linear Accelerator (LINAC) head that may be used with the radiation dose monitoring system of FIG. 1A.

FIG. 2 is a block diagram of an example embodiment of an implementation of the radiation dose monitoring system of FIG. 1A.

FIG. 3A is a block diagram of an example embodiment of a method of monitoring radiation dose using an Artificial Neural Network (ANN).

FIG. 3B is a block diagram of an example embodiment of a method of training the ANN used in the method of FIG. 3A.

FIG. 3C is a block diagram of an example embodiment of a Multi-Layer Perceptron (MLP) Artificial Neural Network (ANN).

FIG. 4A is an example of an image of an example treatment field.

FIGS. 4B-4D are images of certain features of radiation field segments of the example treatment field of FIG. 4A.

FIGS. 5A-5B are plots showing the errors during training of a multilayer perceptron (MLP) using data from a Varian TrueBeam device and an Elekta Agility device, respectively.

FIG. 6A is a plot showing the correspondence between calculation and measurement during MLP training on a Varian TrueBeam device.

FIG. 6B is a plot showing percentage error by effective primary field size during MLP training on a Varian TrueBeam device.

FIG. 7A is a plot showing the correspondence between calculation and measurement during MLP training on an Elekta Agility device.

FIG. 7B is a plot showing percentage error by effective primary field size during MLP training on an Elekta Agility device.

FIGS. 8A-8B are histograms comparing error distribution in training and validation on a Varian TrueBeam device.

FIGS. 8C-8D are histograms comparing error distribution in training and validation on an Elekta Agility device.

FIGS. 9A-9C are graphs showing segment error for Volumetric Modulated Arc Therapy (VMAT) fields.

FIGS. 10A-10B are plots showing modelling error depending on the number of hidden nodes used in the ANN from data obtained for the Varian TrueBeam and Elekta Agility devices, respectively.

FIGS. 11A-11B are plots comparing MLP error with the error for an analytic model for the Varian TrueBeam and Elekta Agility devices.

FIGS. 12A-12B are plots comparing measured error with calculated error for different MultiLeaf Collimator (MLC) and Monitor Unit (MU) sizes.

FIGS. 13A-13B are images showing examples of a composite field constructed from QA fields and AOF fields for the Elekta Agility device.

FIGS. 14A-14C are schematic diagrams of example embodiments of different sensor configurations that may be used by the radiation dose monitoring system.

FIG. 15 shows a schematic diagram of an example embodiment of a two-dimensional (2D) detector array that may be used with the radiation dose monitoring system of FIG. 1A.

FIG. 16 shows an example of an image of an example treatment field, beside which are example images of certain example features of radiation field segments.

FIG. 17 shows a histogram showing the difference (% error) between measured and ANN predicted signals.

FIG. 18 shows a 3D plot showing measured and ANN predicted signals.

FIG. 19 shows the measurement and ANN predicted signals of a large field with Gamma analysis.

FIG. 20 shows the measurement and ANN predicted signals of a clinical IMRT field segment with Gamma analysis.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide examples of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements or steps. In addition, numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the example embodiments described herein.

It should also be noted that the terms “coupled” or “coupling” as used herein can have several different meanings depending in the context in which these terms are used. For example, the terms coupled or coupling can have a mechanical or electrical connotation. For example, as used herein, the terms coupled or coupling can indicate that two elements or devices can be directly connected to one another or connected to one another through one or more intermediate elements or devices via an electrical or magnetic signal, electrical connection, an electrical element or a mechanical element depending on the particular context. Furthermore, certain coupled electrical elements may send and/or receive data.

Unless the context requires otherwise, throughout the specification and claims which follow, the word “comprise” and variations thereof, such as, “comprises” and “comprising” are to be construed in an open, inclusive sense, that is, as “including, but not limited to”.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

Similarly, throughout this specification and the appended claims the term “communicative” as in “communicative pathway,” “communicative coupling,” and in variants such as “communicatively coupled,” is generally used to refer to any engineered arrangement for transferring and/or exchanging information. Examples of communicative pathways include, but are not limited to, electrically conductive pathways (e.g., electrically conductive wires, electrically conductive traces), magnetic pathways (e.g., magnetic media), optical pathways (e.g., optical fiber), electromagnetically radiative pathways (e.g., radio waves), or any combination thereof. Examples of communicative couplings include, but are not limited to, electrical couplings, magnetic couplings, optical couplings, radio couplings, or any combination thereof.

Throughout this specification and the appended claims, infinitive verb forms are often used. Examples include, without limitation: “to detect,” “to provide,” “to transmit,” “to communicate,” “to process,” “to route,” and the like. Unless the specific context requires otherwise, such infinitive verb forms are used in an open, inclusive sense, that is as “to, at least, detect,” to, at least, provide,” “to, at least, transmit,” and so on.

A portion of the example embodiments of the systems, devices, or methods described in accordance with the teachings herein may be implemented as a combination of hardware or software. For example, a portion of the embodiments described herein may be implemented, at least in part, by using one or more computer programs, executing on one or more programmable devices comprising at least one processing element, and at least one data storage element (including volatile and non-volatile memory). These devices may also have at least one input device (e.g., a keyboard, a mouse, a touchscreen, and the like) and at least one output device (e.g., a display screen, a printer, a wireless radio, and the like) depending on the nature of the device.

It should also be noted that there may be some elements that are used to implement at least part of the embodiments described herein that may be implemented via software that is written in a high-level procedural language such as object-oriented programming. The program code may be written in C, C++ or any other suitable programming language and may comprise modules or classes, as is known to those skilled in object-oriented programming. Alternatively, or in addition thereto, some of these elements implemented via software may be written in assembly language, machine language, or firmware as needed.

At least some of the software programs used to implement at least one of the embodiments described herein may be stored on a storage media (e.g., a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions, such as program code, for one or more processors. The program code may be preinstalled and embedded during manufacture and/or may be later installed as an update for an already deployed computing system. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

The present disclosure provides systems and methods for quality assurance in the field of radiation treatment and in particular to monitoring that can be used for the real-time, off-line, pre-treatment or post-treatment quality assurance verification of the delivery of radiation dose. The present disclosure provides a discussion of such systems and methods, including theory and experimental data, which is meant to aid the user in understanding these innovations and is not intended to be limiting.

A RQCS conventionally monitors a treatment beam by comparing a radiation sensor device output with a predicted signal calculated by an analytic numerical model based on the physics of the beam geometry, treatment unit characteristics, and detector unit characteristics for the radiation system being monitored. The RQCS may take measurements in advance of treatment or they can be monitoring during treatment. The radiation sensor device may be based on a well-understood radiation interaction and response by a large area gradient ionization chamber and generally performs well. However, the analytical model requires many physical and empirical parameters. A large number of complex dosimetry measurements are required to derive and fine tune these parameters for each beam energy and for a type of medical linear accelerator, which is laborious and requires a substantial amount of time and effort to perform.

The inventors have also determined that the performance of the analytical model can be improved to handle some unusual beam geometrical situations as well as other challenging situations. For example, some challenges addressed by the teachings of the present disclosure may include, among others, at least one of: (1) predicting an RQCS signal in highly irregular beam geometries in order to make verification of dose delivery as precise as possible and to maintain the same therapeutic dose to the target while simultaneously minimizing radiation dose delivery to regions surrounding the target (e.g. organs), (2) developing an analytical model in an accurate and timely manner when new radiation system are used, and (3) real-time monitoring of daily fractional doses since in some cases the performance of the analytic method may not be robust enough to be used in real-time monitoring, i.e. when a patient is on the treatment couch, and a quick decision has to be made. Furthermore, small variations in the RQCS radiation sensor and the radiation source may lead to sub-optimal results in the performance of the analytical model.

In one aspect, in accordance with the teachings herein, there is provided at least one example embodiment of a computer implemented method for predicting a radiation monitoring signal (e.g. the RQCS signal) based on using an artificial neural network (ANN) engine that comprises at least one ANN that is used to provide predicted radiation measurements. In the description, reference is made to a single ANN but it should be understood that there are embodiments and/or situations in which more than one ANN is used, as is described in further detail below.

The use of an ANN may be more robust in that it provides a less time-consuming way to model the radiation source and radiation detector by learning from actual data and it is more robust in any variation in the RQCS radiation sensor and the radiation source. This is very important since it may take the ANN several hours to be trained to accurately predict radiation measurements whereas for analytical models it can take days or weeks to accurately model a new or updated radiation system so that the analytical model provides accurate estimates of radiation dose.

The ANN may be implemented using a multilayer perceptron (MLP) that is trained using a supervised learning technique by mapping input features of radiation fields to known measurement outputs. An MLP consists of multiple layers of neurons (referred to as nodes), which have a nonlinear computational unit and are fully connected to each other in a parallel and distributed manner. It should be noted that in other embodiments other types of ANNs can be used such as, but not limited to, convolutional neural networks, as is described further below. An MLP can be used to model a well-defined physical system.

Based on the design specification of the radiation monitoring sensor, also known as a radiation sensor, and the radiation treatment unit geometry, several features can be defined that are used by the ANN to more accurately predict or simulate the radiation monitoring signal. In some embodiments, the radiation monitoring sensor can be a large area gradient ion radiation sensor which has at least one spatially sensitive large-area ionization chamber (with a 1-D gradient per ionization chamber) that is placed in the path of the radiation beam. Examples of the large area gradient ion radiation sensor are provided in U.S. Pat. No. 8,119,978, and U.S. patent application publication number 2018/0172845 which are each hereby incorporated by reference in their entirety. For example, in at least one embodiment described herein, the features used by the ANN include features that describe the fluence of the radiation beam segment, the detector sensitivity, and certain linear accelerator beam characteristics.

Reference radiation fields developed for QA and AOF measurement may be used for training of the ANN. For example, in the study discussed herein, more than 300 IMRT segments from a few head-and-neck and prostate plans were randomly selected for training (80%) and validation (remaining 20%). In order to avoid possible overfitting and lack of regularization and loss of generalization of the ANN, the performance of the ANN may be evaluated for different numbers of hidden nodes when the ANN is implemented using an MLP.

As previously mentioned, the RQCS may comprise a radiation sensor that is a large-area position-sensitive ion chamber, as well as a barometer, a thermometer, and an inclinometer. In one implementation, the device has a sensitivity gradient along the multileaf collimator (MLC) direction, which in one example implementation, may be achieved by a small slope (˜3°) in two electrode plates which changes the thickness of the active volume.

The RQCS radiation sensor attaches to the accessory tray holder of a linear accelerator and connects wirelessly to a transceiver using a Bluetooth interface. Charge collected in the radiation sensor during treatment beam delivery is corrected for temperature and pressure using the on-board barometer and thermometer. Gantry and collimator angle are measured at the same time using the integrated inclinometer. The amount of charge and the gantry/collimator angles are digitized and reported to RQCS data management software. The software is interfaced with a linear accelerator and accesses patient-specific treatment information. This allows comprehensive treatment monitoring by displaying information of the patient to be treated, the treatment beam being delivered, the reference count, and the measured count for each beam segment in real-time. The reference count can be established by measurement during patient-specific quality assurance, or it can be calculated using a detector response calculation module such as IQM_Calc (Islam et al., 2009). The calculation module consists of analytic functions that calculate the output signal using an element-wise integration technique, which incorporates MLC dosimetric parameters and the spatial response of the gradient ion chamber of the radiation sensor (Islam et al, 2009). The operation of a RQCS that uses an analytic numerical calculation module is further described in U.S. Pat. No. 8,119,978, which is hereby incorporated by reference in its entirety.

Precise characterization of detector response and its numerical modeling requires laborious measurement, data processing, and optimization of numerous parameters and constants in the equations, which may take on the order of days, weeks or months. However, the inventors have determined that the functional relationship between the beam geometry (input) and the RQCS count (i.e. the radiation sensor output) may be modeled using an ANN, such as an MLP, where the beam geometry is provided as an input and the RQCS count is generated as an output of the ANN. The modeling using an ANN can be done in a few hours or a day or so, which is much shorter than the time needed to develop or update an analytical model. The MLP is a universal function approximator (Irie et al., 1988; Hetcht-Nielsen et al., 1989; Hornik et al., 1989).

However, one challenge in using an ANN is determining the features that should be used by the ANN which allow it to accurately predict the radiation detection signal (i.e. the RQCS detection signal for the RQCS). In accordance with the teachings herein, the inventors have determined that these features may include beam fluence in the form of a 2D image that may be characterized using a certain number of features such as, but not limited to, up to 10 features for example, by considering primary and secondary intensity moments. The number of features that are used may depend on the particular type of radiation sensor configuration that is used. Advantageously, rather than feeding the ANN with a large amount of input data (e.g. pixel by pixel values of a 2D image), the inventors have discovered that a small set of features may be provided to the ANN which allows for a significant reduction in the input size, more efficient training of the ANN and faster operation by the ANN which allows for real-time operation as well as off-line, pre-treatment or post-treatment quality assurance.

It should be noted that the ANN-based radiation quality monitoring system and related methods can be adapted for use with various types of radiation monitoring systems by varying the set of features that are used to train and operate the ANN, as well as potentially varying the topology that is used for the ANN. For example, the radiation sensor that is used by other radiation monitoring systems may operate differently and provide a different number of outputs compared to a gradient ion chamber radiation sensor which therefore requires adjustments to be made by the ANN engine in terms of the type and/or number of ANNs that are used, as well as possibly the number of type of features which are used, which will be described in further detail below.

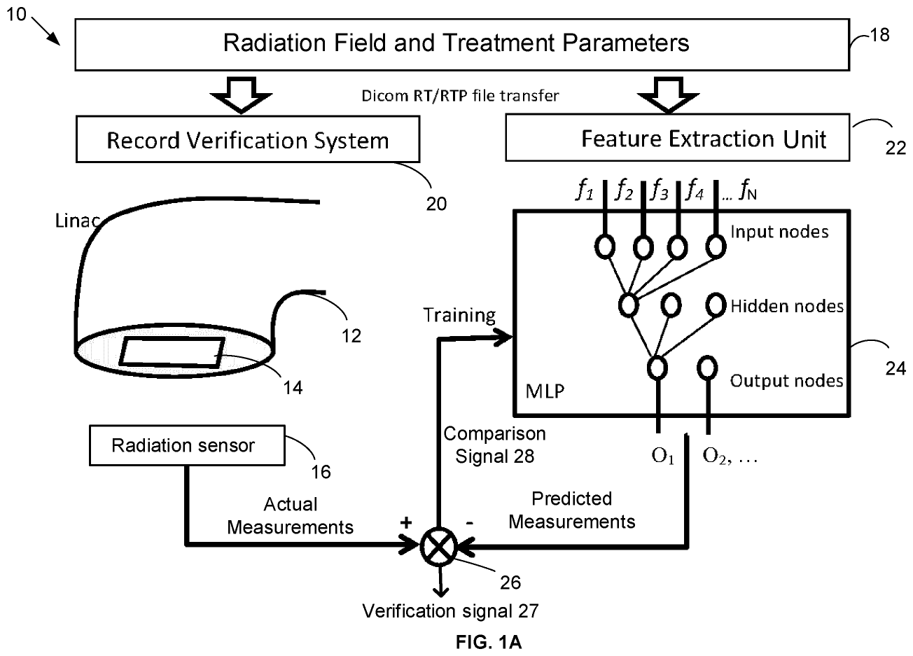

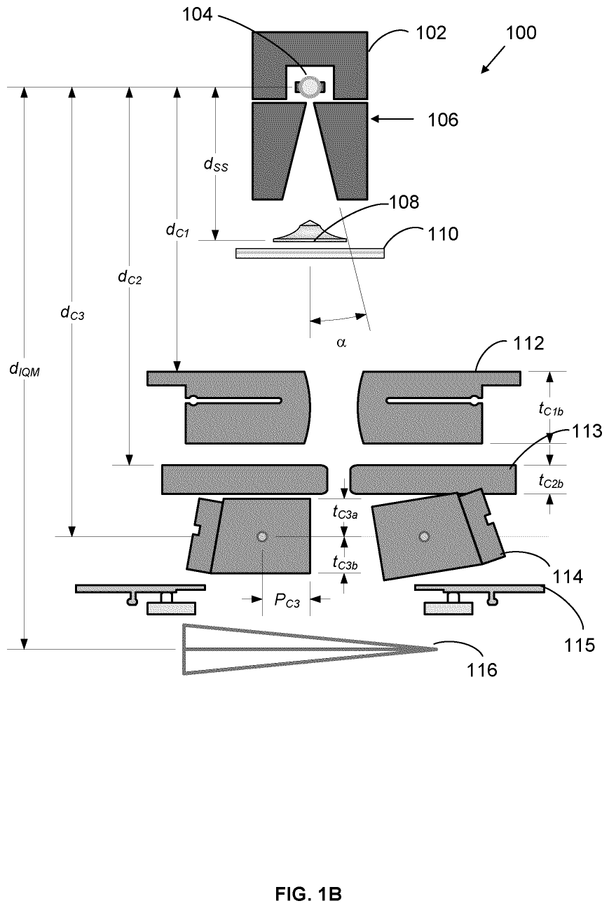

Referring now to FIG. 1A, shown therein is a schematic diagram of the components of an example embodiment of a radiation dose monitoring system 10. The radiation dose monitoring system 10 includes radiation field and treatment parameters 18 that provide information about the patient to be treated and the planned treatment parameters including the dose and timing for the generation of the radiation field. The radiation field and treatment parameters 18 are sent to a record verification system 20 which records the values for the radiation field and treatment parameters and verifies that they result in a safe radiation dose. The record verification system 20 provides information to a radiation source 12 to instruct it on how to generate radiation beams to treat the patient.

The radiation source 12 delivers the radiation beams through an MLC 14 that causes the radiation beams to have an intensity that varies with location to create a treatment beam with a predefined geometry for treating a certain volume of the patient (not shown). In the example embodiment, the radiation source 12 is a linear accelerator (Linac). In other embodiments, the radiation source 12 can be a proton beam therapy device, examples of which include cyclotrons and synchrotrons or a brachytherapy device. Alternatively, in other embodiments a different type of treatment source can be used such as an acoustic generator, a light generator or a pressure generator. Systems which use these different sources can also be modelled using at least one ANN to generate predicted dose measurements.

Referring now to FIG. 1B, shown therein is an example embodiment of a LINAC head 100. The LINAC head 100 comprises a housing 102 with a primary radiation point source 104 that is bombarded with high energy electrons to generate X-rays which are then shaped into a radiation beam using a primary collimator 106. The radiation beam then passes through a flattening filter 108 and a sensor 110. The flattening filter 108 further shapes the radiation beam so that the radiation beam has a more uniform intensity profile and as a secondary radiation source. The sensor 110 is used by the manufacturer of the radiation source 102 for monitoring purposes prior to beam geometry shaping by the collimators. After the sensor 110, the radiation beam travels through a secondary collimator 112 which has leaves that have a curved face in the x-direction and travel in a horizontal straight line in the x-direction. The radiation beam then travels through the level of back-up jaws 113 that are a certain distance behind the last position of the tips of the MLC leaves. The secondary collimator 112 also has a divergence matched edge 114 in the y-direction (perpendicular to the plane of the page) which pivots about a pivot point. The bottom of the LINAC housing 115 is beneath the divergence matched edge 114. A radiation sensor 116 may then be placed underneath the opening of the LINAC collimator housing 115 to measure the amount of radiation projected to the patient for treatment.

Referring again to FIG. 1A, after the MLC 14, the radiation beam encounters radiation sensor 16, which provides an actual radiation measurement. In various embodiments, the radiation dose monitoring system 10 may include different types of radiation sensors such as a large area gradient ion radiation sensor, a single detector, a line of detectors, an array of line detectors, a 2D array of small ion chamber detectors, a 2D array of solid state detectors (e.g. diodes) for measuring the intensity of the radiation beams and a 3D array of radiation detectors. The radiation sensor 16 may be a transmission detector where the radiation beam travels through the radiation sensor which sits between the patient and the LINAC (e.g. on the LINAC head as shown in FIG. 1B), or the radiation sensor 16 may not be a transmission detector and does not sit between the LINAC head and the patient. In the case of a 3D array of radiation detectors, the radiation detectors may be configured in a cylindrical geometry, a cube geometry, or a parallelepiped geometry. In different embodiments, one or more radiation sensors may be placed in the primary beam, outside of the primary beam, or a combination thereof. In other embodiments, the radiation sensor 16 can be located immediately downstream of the collimator opening, or further away from the collimator opening and before the patient, or the radiation sensor 16 can be located downstream of the patient to measure the radiation beam after it has passed through the patient (i.e. for exit beam monitoring) or elsewhere in the vicinity of the LINAC (i.e. to detect radiation from scatter or reflection from the patient or other objects in the room). For the case where the radiation sensor 16 is not located immediately downstream of the collimator opening, other input features may also be needed for the operation of the ANN such as the patient's geometry at the treatment region, and the location of the patient on the treatment table/couch (both of these features can be derived from CT scan data of the patient, which is usually readily available). In some embodiments, the radiation detection can occur off-line and radiation can be measured prior to the treatment of a patient in a simulated or practice session; while in other embodiments the detection of radiation can occur in real-time during the treatment of a patient.

The radiation dose monitoring system 10 includes a feature extraction unit 22 that determines values for certain features related to the radiation field and treatment parameters 18. The extracted feature values, which are symbolized as f₁ to f_{N}, are entered into an ANN 24. In this example embodiment, the ANN 24 is an MLP but other types of ANNs can be used in other embodiments. The feature extraction unit 22 and the ANN 24 can be implemented using a processor. The ANN 24 produces at least one output, such as O₁ and O₂, that corresponds to the number of different measurements made that are generated by the radiation sensor 16 for a given time sample. The output of the ANN 24 is referred to as a predicted radiation measurement. In this case, the radiation sensor 16 has one output that varies over time referred to as the actual radiation measurement and the ANN 24 also has one output that varies over time referred to as the predicted radiation measurement. In other embodiments, where different radiation sensors are used, the output produced by the ANN 24 can be a one-dimensional array of outputs O₁ to Oₙ, a two-dimensional array of outputs O₁₁ to Oₘₙ, a three-dimensional array of outputs O₁₁₁ to Oₘₙₒ, or some other suitable arrangement of outputs that correspond to the different outputs produced by the radiation sensor.

The actual radiation measurement and the predicted radiation measurement are compared by a comparator 26. The comparator 26 then produces a verification signal 27 and a comparison signal 28 that can be used for many purposes. The verification signal 27 and the comparison signal 28 can be produced in a similar fashion such as how the comparison signal 28 is generated in FIG. 1A. Alternatively, only one of signals 27 and 28 may be generated but used for each of the purposes described for both signals 27 and 28. For example, the comparison signal 28 may be used for training of the ANN while the verification signal 27 can be used for at least one of user notification and radiation source control. Accordingly, the accuracy and safety of beam delivery in radiation therapy can be validated in real time.

For example, in some embodiments, the comparison signal 28 is used for training the ANN 24. This is done by interpreting the comparison signal 28 as representing errors between the actual radiation measurements and the predicted radiation measurements. The ANN 24 then uses the errors provided by the comparison signal to adjust the weights of at least one node in at least one of the input nodes, the hidden nodes and the output nodes so that the error of future predicted radiation measurements when compared to corresponding future actual radiation measurements are smaller. For example, the weights of the nodes can be updated using a gradient descent method which back-propagates from the output nodes to the hidden nodes and then from the hidden nodes to the input nodes.

As another example, in some embodiments, the verification signal 27 is used to send a notification to the user of the radiation dose monitoring system 10 to indicate whether the radiation dose that is being delivered is within a safe range of the radiation dose specified in the treatment plan.

As another example, in some embodiments, the verification signal 27 is used to directly control the operation of the radiation source 12. For example, when the verification signal 27 indicates that the radiation dose being delivered by the radiation source 12 is within a safe range of the radiation dose specified in the treatment plan, then the verification signal 27 is used to further allow or enable the radiation source 12 to continue to generate and deliver the radiation treatment beam. Conversely, when the verification signal 27 indicates that the radiation dose being delivered by the radiation source 12 is not within a safe range of the radiation dose specified in the treatment plan, then the verification signal 27 may be used to generate a control signal that is provided to the radiation source 12 to disable or stop the radiation source 12 so that it can no longer generate and deliver the radiation treatment beam.

Alternatively, in some embodiments, when the verification signal 27 indicates that the radiation dose being delivered by the radiation source 12 is not within a safe range of the radiation dose specified in the treatment plan, then the verification signal 27 is used to generate a control signal that is provided to the radiation source 12 to adjust the amount of radiation in the radiation beam that is generated by the radiation source 12 so that the amount of radiation in the treatment beam is within safe operating limits or the amount of radiation in the radiation treatment beam is within an acceptable predefined range of the amount of radiation that has been prescribed for the radiation treatment session.

Alternatively, in some embodiments, when the verification signal 27 indicates that the radiation dose being delivered by the radiation source 12 is not within a safe range of the radiation dose specified in the treatment plan, then the verification signal 27 is used to generate a control signal that is provided to the radiation source 12 to either disable or stop the operation of the radiation source 12 or to adjust the amount of radiation that is generated by the radiation source 12 as described previously.

Alternatively, in some embodiments, when the verification signal 27 indicates that the radiation dose being delivered by the radiation source 12 is not within a safe range of the radiation dose specified in the treatment plan, then the verification signal 27 may be used to display a graphical user interface (GUI) with an output message to an operator of the radiation source 12 that the radiation source 12 is not within a safe range of the radiation dose specified in the treatment plan. The GUI may include at least one of a first input option to allow the user to stop the operation of the radiation source 12 and a second input option to allow the user to modify the operation of the radiation source 12 so that it is operating with the safe range of the radiation dose specified by the treatment plan.

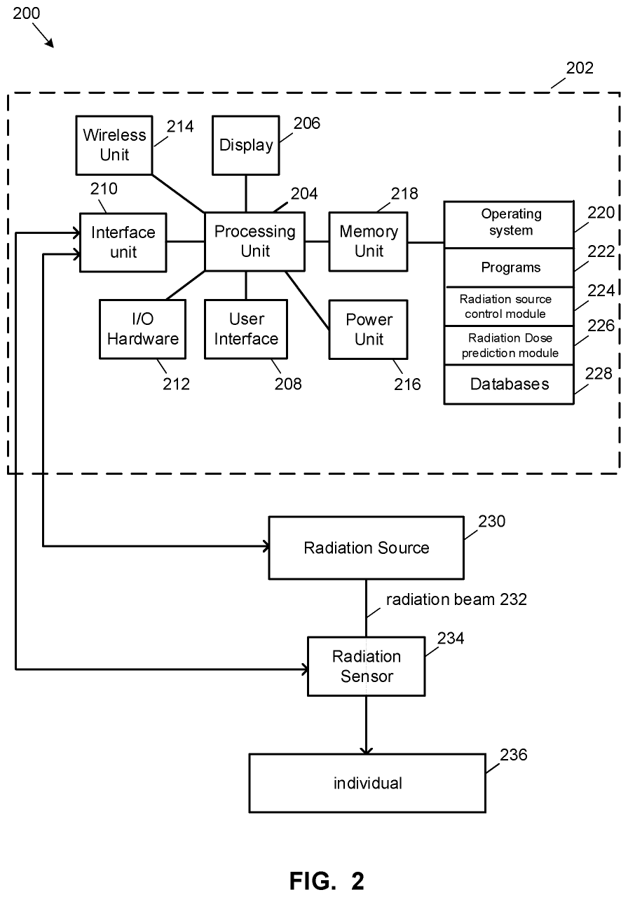

Referring now to FIG. 2, shown therein is a block diagram of the components of an example embodiment of a radiation dose monitoring system 200 that can be used to monitor the amount of radiation being provided to a patient in accordance with the teachings herein. The radiation dose monitoring system 200 is used with a radiation source 228 and the system 200 includes an operator unit 202, and a radiation sensor 234. The radiation source 230 generates a radiation beam 232 to provide radiation to a volume (i.e. treatment volume) of an individual 236 (i.e. a patient) that requires radiation treatment. The radiation sensor 234 is used to measure the amount of radiation that is in the radiation beam that is directed to the individual 236. The radiation source 230 is similar to the radiation source 12, which was described previously. The radiation sensor 234 is similar to the various radiation sensors described with relation to FIG. 1A.

In general, a user may interact with the operator unit 202 to perform at least one of quality assurance on the radiation source 230, to first train at least one ANN that is used to predict the radiation dose provided by the radiation source 230 and to ensure that the radiation delivered to the individual 236 is within an acceptable level of the radiation treatment parameters. After training, the ANN can be used in real-time during actual delivery of radiation to the individual 236 or it may be used during off-line, pre-treatment or post-treatment quality assurance. The system 200 is provided as an example and there can be other embodiments of the system 200 with different components or a different configuration of the components described herein.

The operator unit 202 comprises a processing unit 204, a display 206, a user interface 208, an interface unit 210, Input/Output (I/O) hardware 212, a wireless unit 214, a power unit 216 and a memory unit 218. The memory unit 218 comprises software code for implementing an operating system 220, various programs 222, a radiation source control module 224, a radiation dose prediction module 226, and one or more databases 228. Many components of the operator unit 202 can be implemented using a desktop computer, a laptop, a mobile device, a tablet, and the like.

The processing unit 204 controls the operation of the operator unit 202 and the radiation source 230. The processing unit 204 can be any suitable processor, controller or digital signal processor that can provide sufficient processing power depending on the configuration, purposes and requirements of the system 200 as is known by those skilled in the art. For example, the processing unit 204 may be a high performance general processor. In alternative embodiments, the processing unit 204 may include more than one processor with each processor being configured to perform different dedicated tasks. In alternative embodiments, specialized hardware can be used to provide some of the functions provided by the processing unit 204.

The display 206 can be any suitable display that provides visual information depending on the configuration of the operator unit 202. For instance, the display 206 can be a cathode ray tube, a flat-screen monitor and the like if the operator unit 202 is a desktop computer. In other cases, the display 206 can be a display suitable for a laptop, tablet or handheld device such as an LCD-based display and the like. The display 206 can provide notifications to the user of the radiation dose monitoring system 200.

The user interface 208 can include at least one of a mouse, a keyboard, a touch screen, a thumbwheel, a track-pad, a track-ball, a card-reader, voice recognition software and the like again depending on the particular implementation of the operator unit 12. In some cases, some of these components can be integrated with one another. The user interface 208 can receive control inputs from the user for controlling the radiation dose monitoring system 208.

The interface unit 210 includes hardware that allows the processing unit 204 to send and receive data to and from the radiation source 230 and the radiation sensor 234. Accordingly, the interface unit 210 may include analog to digital converters (ADCs) and digital to analog converters (DACs). For example, the processing unit 204 may send control data to the radiation source 230 and receive status data on the operational status of the radiation source 230. The interface unit 210 also receives actual radiation measurements from the radiation sensor 234.

Signal processing hardware may be included in the interface unit 210 or as a separate preprocessing unit (not shown) in order to pre-process the actual radiation measurements. The preprocessing that is done may include standard signal processing techniques such as, but not limited to, at least one of amplification, filtering and de-noising (e.g. averaging) using parameters that can be determined from experimentation as is known by those skilled in the art.

The interface unit 210 may also include other interfaces that allow the operator unit 202 to communicate with other devices or computers. In some cases, the interface unit 208 can include at least one of a serial port, a parallel port or a USB port that provides USB connectivity. The interface unit 210 can also include at least one of an Internet, Local Area Network (LAN), Ethernet, Firewire, modem or digital subscriber line connection. Various combinations of these elements can be incorporated within the interface unit 210.

The I/O hardware 212 is optional and can include, but is not limited to, at least one of a microphone, a speaker and a printer, for example. Accordingly, the I/O hardware 212 can provide the processing unit 204 with other ways that it can receive input or provide output, such as via an audio device (not shown).

The wireless unit 214 is optional and can be a radio that communicates utilizing CDMA, GSM, GPRS or Bluetooth protocol according to standards such as IEEE 802.11a, 802.11b, 802.11g, or 802.11n. The wireless unit 214 can provide the processing unit 204 with a way of communicating wirelessly with certain components of the radiation dose monitoring system 200 or with other devices or computers that are remote from the system 200.

The power unit 216 can be any suitable power source that provides power to the various components of the operator unit 202 such as a power adaptor or a rechargeable battery pack depending on the implementation of the operator unit 202 as is known by those skilled in the art.

The memory unit 218 can include RAM, ROM, one or more hard drives, one or more flash drives or some other suitable data storage elements such as disk drives, etc. The memory unit 218 may be used to store an operating system 220 and programs 222 as is commonly known by those skilled in the art. For instance, the operating system 220 provides various basic operational processes for the operator unit 202. The programs 222 include various user programs so that a user can interact with the operator unit 202 to perform various functions such as, but not limited to, acquiring data, viewing and manipulating data, adjusting parameters for data analysis as well as sending messages as the case may be. The memory unit 218 can also store software instructions for implementing a radiation dose prediction module 224.

The processing unit 204 may access the memory unit 218 to load the software instructions from any of the programs 222 and/or the radiation dose prediction module 224 for executing the software instructions in order to control the radiation dose monitoring system 100 to operate in a desired fashion. The processing unit 204 may also store various operational parameters such as the radiation field and treatment parameters 18, patient data, status data, test parameters, as well as actual radiation measurement data, predicted radiation measurement data, error data for the differences between the actual radiation measurement data and the predicted radiation measurement data and performance data.

The radiation source control module 224 is used to control the operation of the radiation source 230. The radiation source control module 224 comprises software code that when executed, by the processing unit 204 for example, includes instructions for controlling the intensity, waveforms and timing sequence for radiation beams that are to be generated by the radiation source 230. The radiation source control module 224 can obtain data for these instructions in various ways including accessing the databases 228 to determine the individual that will receive the radiation treatment and then obtaining the radiation field and treatment parameters for the individual from the databases 228. Alternatively, or in addition thereto, the user of the radiation treatment system 200 may provide further instructions or modify the instructions by entering control inputs via the user interface 208.

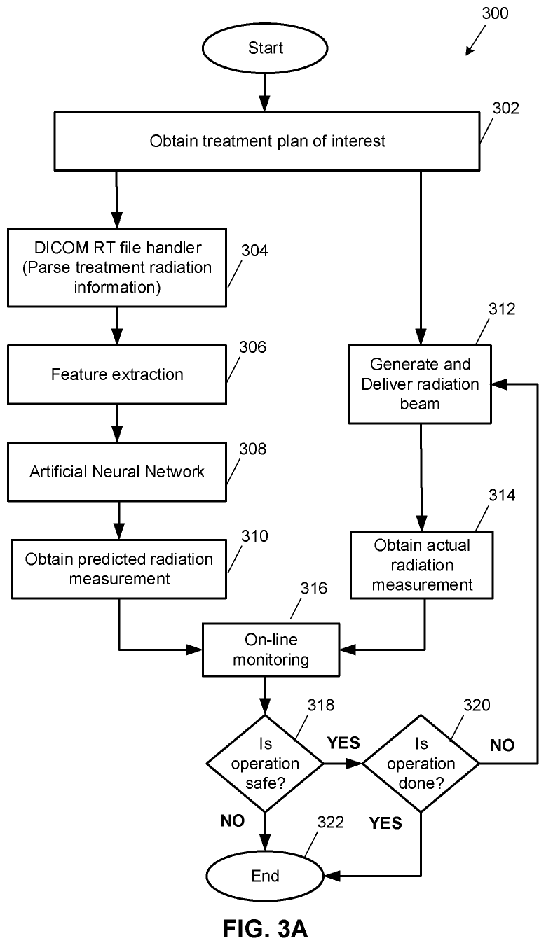

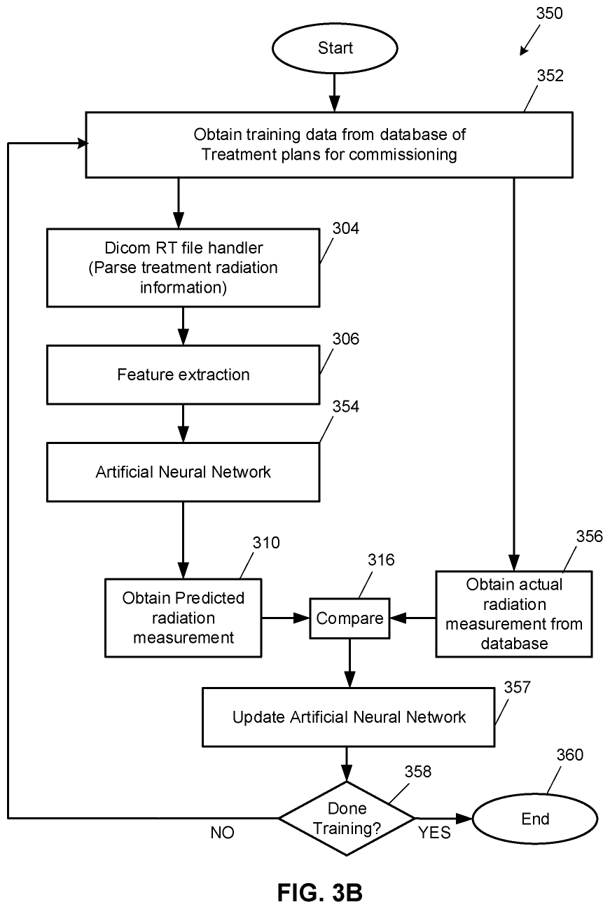

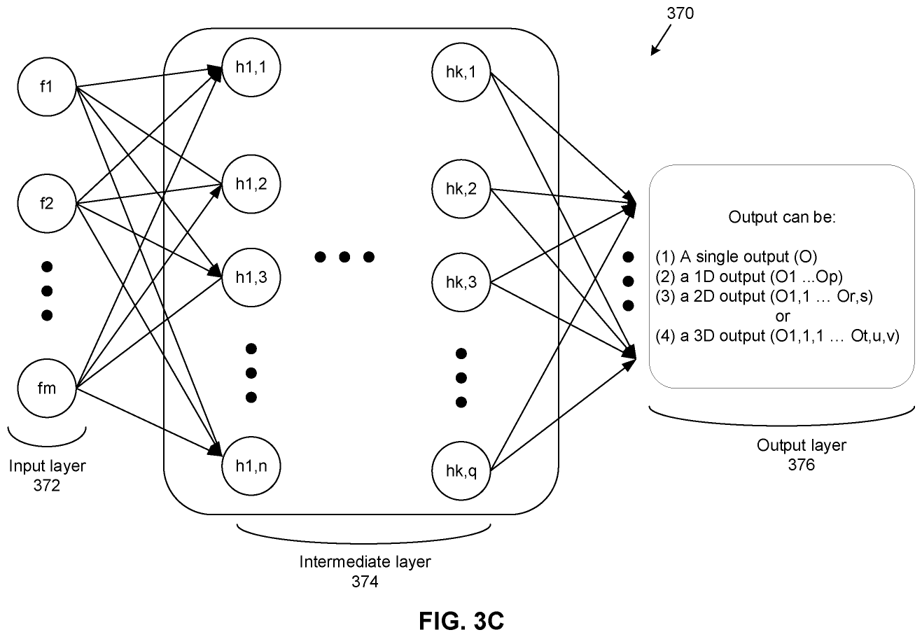

The radiation source control module 224 can also perform quality assurance by working with the radiation dose prediction module 226 which uses an ANN engine to determine predicted radiation measurements. The radiation source control module 224 can analyze the predicted radiation measurements to ensure that the radiation source 230 is operated within predetermined safe limits, that is determined so that an acceptable range of radiation can be provided to the individual during treatment. These predetermined safe limits ensure that the radiation beam 232 is being generated accurately to provide treatment to the target volume of the individual while minimizing radiation exposure to other areas of the individual that do not require radiation treatment. For example, the radiation source control module 224 can control the operation of a radiation dose monitoring method 300, which uses an ANN. An example embodiment of the radiation dose monitoring method 300 is described further in relation to FIG. 3A. The radiation source control module 224 can also perform operations for a method of training the ANN. An example embodiment of an ANN training method 350 is described further in relation to FIG. 3B.

The radiation dose prediction module 226 is used to generate predicted radiation dose measurements, which can be done before or while the radiation source 230 is generating and delivering the radiation beam 232. The radiation dose prediction module 226 employs an artificial neural network (ANN) engine to predict these measurements. The ANN engine uses at least one ANN that is trained, in accordance with the teachings herein, before being used when radiation treatment is provided to the individual 236. In at least one embodiment, after initial training, the ANN that is used by the ANN engine may be re-trained or calibrated at periodic intervals thereafter. Alternatively, in at least one alternative embodiment, the ANN that is used by the ANN engine may be continuously trained (i.e. after each treatment session) even while it is used to perform quality assurance on radiation treatment provided to the individual 236.

The radiation source control module 224 and the radiation dose prediction module 226 are typically implemented using software, but there may be instances in which it is implemented using FPGA or application specific circuitry. For ease of understanding, certain aspects of the methods described in accordance with the teachings herein are described as being performed by the radiation source control module 224 and the radiation dose prediction module 226. However, it should be noted that these methods are not limited in that respect, and the various aspects of the methods described in accordance with the teachings herein may be performed by other modules in other embodiments.

The databases 228 can be used to store data for the system 200 such as system settings, parameter values, and calibration data. The databases 228 can also store other information required for the operation of the programs 222 or the operating system 220 such as dynamically linked libraries and the like. The databases 228 can also store data related to the structure, operation and performance of the ANN used by the radiation dose prediction module 226. For example, in at least one embodiment, the databases 228 may include training data for the ANN, and optionally a history of the errors of the ANN during training and in actual use.

In another embodiment, the databases 228 store several ANNs that have been trained using training data sets obtained when treating the same treatment region of a patient's body, where the weights of the nodes in the ANNs are obtained using a stochastic process. In this case, training is done N times using the training data set to obtain N ANNs, where N is an integer such as, but not limited to, 2<=N<=10. Alternatively, N may be greater than 10. The N ANNs are slightly different since a stochastic process is employed in determining the weights of the ANN, such as for, but not limited to, ANNs that are MLPs, for example. In this case each ANN may be referred to as a child ANN. When obtaining each ANN, characteristics of the ANN can be stored such as the data training sets that were used, the network topology, the network size, training errors and the like. During use, the ANN engine employs the N different ANNs to obtain N different predicted radiation measurements. The predicted radiation measurement from each ANN is then averaged together to obtain a more reliable predicted radiation measurement. The standard deviation of the predicted radiation measurements can be added to the overall estimate of the uncertainty in the predicted radiation measurement.

In another embodiment, the databases 228 store several ANNs that have each been trained using training data sets obtained when treating different treatment regions of a patient's body such as their abdominal region, breast region, head region and the like. In this case, during use, the ANN engine selects the ANN that was trained using training data obtained for the treatment region that the person will be receiving radiation treatment for.

In another embodiment, the databases 228 store several sets of ANNs where the ANNs in each set of ANNs have been trained using training data sets obtained when treating the same treatment region. For example, the training data sets may have been obtained for M treatment regions such as, but not limited to, the head, the breast and the leg, where M is an integer greater than or equal to 2. Each of the M types of training data sets are used to train the ANN N different times, to obtain N ANNs for each type of training data set, where each ANN is slightly different when a stochastic process is employed for determining the weights of the ANN, as explained earlier. During use, the ANN engine selects the N ANNs that were trained using training data that is the same as the treatment region that is to be treated. The N different ANNs are then used by the ANN engine to obtain N different predicted radiation measurements which can then be averaged together to provide an averaged predicted radiation measurement. The standard deviation of the predicted radiation measurements can be used to estimate the uncertainty in the predicted radiation measurement.

The operator unit 202 comprises at least one interface that the processing unit 204 communicates with in order to receive or send information. This interface can be the user interface 208, the interface unit 210 or the wireless unit 214. For instance, some of the various operational and/or calibration parameters used by the system 200 may be inputted by a user through the user interface 208 or they may be received through the interface unit 208 from a computing device. The processing unit 204 can communicate with either one of these interfaces as well as the display 206 or the I/O hardware 212 in order to output information related to one or more of radiation treatment monitoring, the operation of the radiation source 228 and the effectiveness of the radiation treatment. In addition, users of the operator unit 202 can communicate information across a network connection to a remote system for storage and/or further analysis in some embodiments. This communication may also include email communication.

Referring now to FIG. 3A, shown therein is a flowchart of an example embodiment of the radiation dose monitoring method 300. The radiation dose monitoring method 300 can be used during QA testing of a radiation system, such as radiation system 200, when the individual 236 who will receive radiation treatment can be replaced by a phantom. The radiation dose monitoring method 300 is also used when radiation is being delivered to the individual 236 to determine if the radiation is being delivered according to the treatment plan and to make sure that the radiation source 228 is operating within safe limits.

At act 302, the method 300 accesses data for the treatment plan of interest, which may be in the form of a DICOM RT file or another electronic patient record format. The treatment plan of interest includes test treatment plan data that is used during QA testing of the radiation system 200 and the radiation source 228. During actual use with the individual 236, the treatment plan of interest includes the actual treatment parameters for the particular individual 236 that will receive the radiation treatment. The data can be accessed from the databases 228 or some other memory device. Alternatively, this data can be inputted by the user via the user interface 208 or received from a remote device via the interface unit 210 or the wireless unit 214, for example.

At act 304, the treatment plan data is parsed to obtain radiation treatment field (geometry and radiation intensity) data. The DICOM RT file may be parsed using a routine from MATLAB™, such as Dicomread which is available in the MATLAB default library, or another suitable program as is known by those skilled in the art. Alternatively, the treatment plan data can have an RTP, ARIA® or Suitestensa RT file format and a person of skill in the art can write a program to parse such file formats. For example, the RTP format includes text which can be parsed to obtain the treatment plan data including machine type, the x and y positions of the jaws and the position of the MLC.

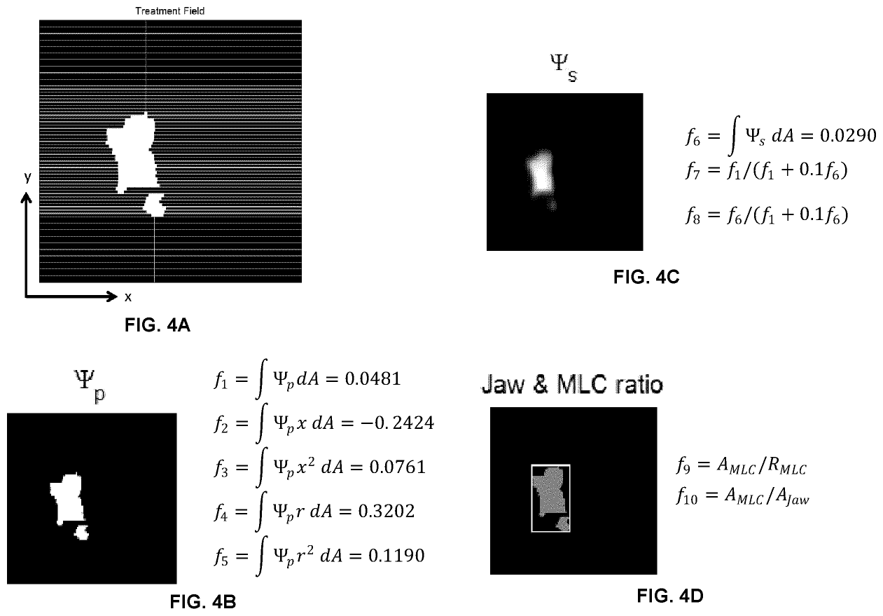

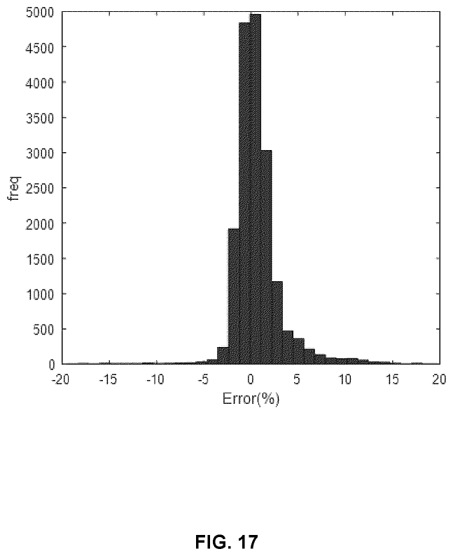

At act 306, feature extraction is performed on the radiation treatment field data in order to obtain values for the features that are used as the inputs to the ANN. The feature extraction may be based on the particular type of sensor 16, 116 or 234 that is used for measuring the radiation dose. Examples features are described with respect to FIGS. 4A-4D and 17.

At act 308, the feature values that were extracted at act 306 are provided as inputs into the ANN. The ANN is then operated to determine the predicted radiation measurement. In at least some embodiments, the ANN may be an MLP but other types of neural networks can be used in other embodiments. The input of the ANN includes the radiation treatment field geometry (e.g. features), and the output is the radiation sensor readings. In alternative embodiments, there may be M ANNs that were trained for M treatment regions and the ANN that is trained for the treatment region that is currently being treated is selected. Alternatively, there may be N ANNs that were trained for the treatment region that is being treated and the N ANNs are each operated to provide N intermediate predicted radiation measurements which are then averaged to provide the predicted radiation measurement.

At act 310, the predicted radiation measurement that is provided by the ANN is obtained and stored in memory. It should be noted that the predicted radiation measurement may be obtained before or during the time of actual treatment delivery.

At act 312, operating parameters from the treatment plan of interest are used to begin treatment and generate a radiation beam that is then directed to a phantom during QA testing or to a subject during actual radiation treatment.

At act 314, the actual radiation measurement is obtained from the radiation sensor and stored in memory.

At act 316, the predicted radiation measurements and actual radiation measurements are compared to one another to determine if the radiation system is delivering the expected amount of radiation dose based on the radiation treatment parameters and the prediction that is done by the ANN. These error results can then be used for a number or purposes, such as notifying the user of the comparison results and/or controlling operation of the radiation source.

For example, at act 318, during radiation treatment, it is determined when the difference (i.e. error) between the actual radiation measurements and the predicted radiation measurements are within safe limits. If this is true then the method 300 proceeds to act 320. It is then determined at act 320 whether the radiation treatment is done. If this condition is true then the method 300 proceeds to act 312 and the radiation treatment is continued. Otherwise when the condition at act 320 is false the method 300 proceeds to act 322 and the method 300 and the radiation treatment ends. Alternatively, when it is determined at act 316 that the difference between the actual radiation measurements and the predicted radiation measurements are not within safe limits then the method 300 proceeds to act 320 where radiation treatment is ended.

Referring now to FIG. 3B, shown therein is a block diagram of an example embodiment of a method 350 of training an ANN. The method of training the ANN may be largely automated, and identical for various beam energies and different medical linear accelerator models. The overall time and effort to train and make the ANN ready to use clinically will involve much less time and effort compared to that of the analytic method in existing radiation dosimetry systems. In addition, due to the easy adaptability of the network by training, the ANN may be used to model a specific radiation sensor and linear accelerator pair in each institution for optimal precision and accuracy. In different embodiments, training may be improved using various techniques such as, but not limited to, deep learning with batch normalization and drop-out.

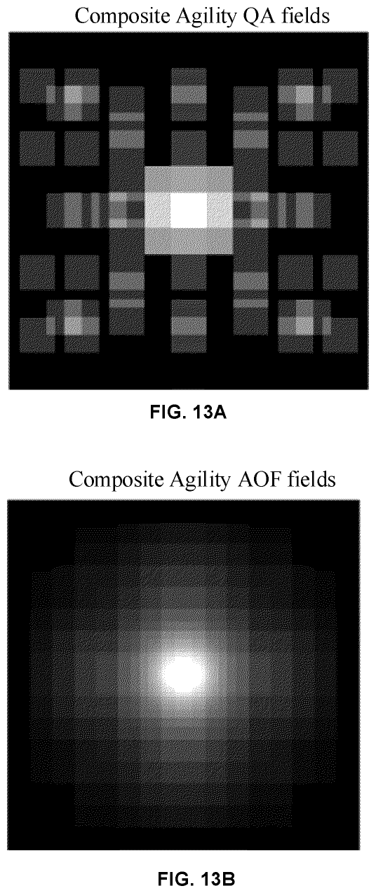

The method 350 begins at act 352 by obtaining electronic records from a database of treatment plans for commissioning, which can be in the form of a DICOM RT file. The training of the ANN is performed by using radiation field data from a variety of treatment plans, extracted from DICOM RT treatment plan files or treatment plan files in other data formats (as explained for method 300), and their corresponding measured chamber signals. The number of fields is large enough to cover the whole area of the radiation detector 234 that is used. Accordingly, a variety of QA and AOF radiation treatment fields may be used, examples of which are shown in FIGS. 13A and 13B. The input data to the ANN includes features quantifying radiation field area, shape, location, intensity; as well as the spatial sensitivity of the ionization chamber.

Act 304 is then performed for parsing the treatment plan data to parse treatment radiation information which is then used for feature extraction at act 306. Acts 304 and 306 can be implemented as described previously for method 300. Values for the input features are then provided to the ANN at act 354 and the ANN is operated to produce predicted radiation measurements. These measurements are then stored at act 310.

At act 356, the actual radiation measurement is obtained from the database. The results of the predicted radiation measurement and the RQCS output measurement 314 are compared at act 316, for example by subtracting one measurement from the other, to determine the resulting errors. These errors are then used to train the ANN at act 357. For example, the error can be backward propagated to adjust the weights of the ANN, thereby strengthening the connections between at least two of the nodes of the ANN. Multiple iterations of error backpropagation may be used to achieve optimal weight distribution so that in use the trained ANN produces outputs with minimum error. Accordingly, the ANN parameters are optimized by minimizing the differences between the predicted and corresponding radiation measurements.

It should be noted that training of the ANN may be accelerated significantly using advanced error back-propagation such as, but not limited to deep learning and co-variance shift. In the development of the ANN discussed herein, basic back-propagation with a fixed learning rate, momentum, and training iterations (number of epochs) was used. The test results, which are discussed in further detail below, showed that a few thousand epochs appear to be sufficient for training without the need for advanced training methods.

At act 358, it is determined whether training is finished. For example, training may be done over a number of iterations, such as up to 20,000 or more, until the error between the predicted radiation measurements and the actual radiation measurements is less than a suitable error amount. If the comparison at act 358 is true then the method 350 proceeds to act 360 where the method 350 is ended. If the condition at act 358 is not true then the method proceeds to act 352 to obtain another training data set and train the ANN for that particular data set.

As described previously, in various embodiments, the training may be done to obtain N different ANNs that are then used in practice to provide a statistically combined amount, such as an averaged result, although other statistical operators may be used such as the median or the trimmed mean, for example. Also, the training can be done to generate M ANNs where each ANN is trained using training data obtained when treating a particular treatment region of the patient (i.e. individual 236).

The development of the ANN, which in this example embodiment is an MLP, for numerical modeling of the radiation sensor 234 is discussed here. Complex problems may require a network with a large number of hidden nodes in multiple hidden layers (deep network) to provide sufficient degrees of freedom. Due to the difficulty of training a deep network, a special learning technique may be used (Hinton et al., 2006). Furthermore, a large-scale input poses another challenge in network training since the number of connections between the input and the hidden nodes as well as the connections between the hidden nodes and the output nodes increase exponentially. For example, if each pixel of an image is used as the input, the number of connections between the input image and one hidden node is more than 262,144 for an image size of 512×512 pixels. A special network with restrictive connections between the layers (e.g. a convolutional network) may be considered in the case of a large-scale multi-dimensional input such as in image processing (Kallenberg et al., 2016). However, the inventors have determined that with a greater understanding of the technical challenges and by carefully selecting input features, an MLP with a small number of hidden nodes is often adequate and efficient for determining predicted radiation measurements.

The following development of the ANN is based on using a large area gradient ion sensor as the radiation sensor (hereafter referred to as the RQCS radiation sensor) and the corresponding treatment unit geometry (i.e. the jaws and the MLC from which the sharp and blurred 2D images shown in FIGS. 4B and 4C were made representing fluence). However, the ANN can be created for different types of radiation sensors and different types of treatment unit geometry. In creating the ANN, 10 features were identified, based on the inventors' research, that allows the ANN to generate predicted radiation measurements with sufficient accuracy (such as less than 3% error for example). The inventors' understanding of the radiation systems and radiation detector physics helps to find appropriate input features such as linear positional sensitivity of the radiation detector, radial response of radiation beam characteristics, and the interplay of the MLCs and the jaws. The inventors purposefully used a smaller number of features to reduce the size of the input data which advantageously avoids the difficulties of large-scale network training.

In order to determine the impact of the input features on the ANN, three different input configurations were tested. These different input configurations included using 5, 8, and 10 features, respectively. The number of hidden layers and the number of hidden nodes govern the degrees of freedom of the ANN, and the optimal size depends on the complexity of the problem. A lack of degrees of freedom hinders precision of the modeling; however, excess degrees of freedom often makes the ANN overfit, which also results in poor prediction accuracy. Each network configuration (3 different input sizes and 4 different hidden node sizes) was simulated 10 times, and each simulation randomly selected 80% of the data for training with the remaining 20% of the data being used for validation.

Referring now to FIGS. 4A-4D, shown therein is an example of an image of a radiation treatment field with corresponding features of radiation field segments. The 10 features were derived from certain fluence measurements obtained for certain areas. As shown in FIG. 4A, treatment beam data such as the Jaw and MLC geometry was extracted from the radiation treatment plans. As shown in FIGS. 4B and 4C, energy fluence maps for primary (Ψₚ) and secondary radiation sources (Ψₛ) were determined using the radio therapeutic properties of the LINAC (i.e. beam geometry data along with measured transmission factors for the jaws and the MLC) in the form of an image with a pixel density of 400×400 at the location of the RQCS radiation sensor. A Gaussian distribution with sigma of 20 mm at the bottom of the flattening filter was assumed to model a secondary source. The radiation produced at the target (i.e. radiation due to primary source) has a high intensity at the center of the treatment field and a diminished intensity at the periphery without the flattening filter. The flattening filter is a piece of metal with a conical shape to compensate for this non-uniformity and is placed right below the radiation source (see FIG. 1B). The flattening filter flattens out the fluence of the radiation beam but a side effect is that it adds a blurred fluence component due to scattering of radiation. The blurred fluence component is an order of magnitude smaller than the fluence due to the primary radiation source. The energy fluences Ψₚ and Ψₛ were determined assuming an isotropic distribution. A radiation source without a flattening filter will have a fluence that is non-uniform (i.e. a non-isotropic distribution).

The spatial variation of energy fluence, mostly in the radial direction, and the positional sensitivity of the RQCS radiation sensor were considered using five features specified in equations (1a) to (1e) shown below.

$$\begin{matrix}{f}_{1} = \int {Ψ}_{p} dA & ( 1 a ) \\ {f}_{2} = \int {Ψ}_{p} xdA & ( 1 b ) \\ {f}_{3} = \int {Ψ}_{p} {x}^{2} dA & ( 1 c ) \\ {f}_{4} = \int {Ψ}_{p} r dA & ( 1 d ) \\ {f}_{5} = \int {Ψ}_{p} {r}^{2} dA & ( 1 e )\end{matrix}$$

As shown in FIG. 4A, x is the direction of the MLC or the direction of detector sensitivity, and y is the orthogonal direction. In addition, r is the radial distance from the center of the treatment field, which is the center of the open field that meets at the isocenter of the treatment unit. When the sensitivity of the chamber in the RQCS radiation sensor is perfectly linear, the two features, f₁ and f₂, are a sufficient representation of the fluence that may be used by the ANN in generating sufficiently accurate prediction radiation measurements.

The feature f₃ may be used to model any possible imperfections or any nonlinearities in the response of the radiation sensor. The sensitivity of the radiation detector in the y direction is neglected for the sake of simulation simplicity and also considering the fact that y directional sensitivity of the RQCS radiation detector is zero in ideal conditions. The spatial variation of a beam characteristic of a linear accelerator (e.g. spatial variation of energy fluence) is considered using features f₄ and f₅ as a function of radial distance from the center of the treatment field. The features f₄ and f₅ take into account a non-ideally flattened radiation beam, and these features can be used to model radiation fluence variance (i.e. when using other radiation sources). In alternative embodiments, if the fluence variation is not a function of the radius from the isocenter of the treatment geometry then other features may optionally be used to account for this behavior. Furthermore, in alternative embodiments, the performance of the ANN may be increased if it is feasible to add more features with higher order such as, but not limited to, xⁿ, rⁿ, with n>2, or if a feature for the y-directional sensitivity is used by the ANN.

The contribution of the secondary radiation source may be modelled by the ANN by using feature f₆ as shown in equation (2a).

$${f}_{6} = \int {Ψ}_{s} dA \qquad ( 2 a )$$

Furthermore, to take into account the relative contribution of the secondary radiation source compared to the primary radiation source, features f₇ and f₈, are included where 10% scatter contribution is assumed (i.e. a scaling factor of 0.1 is applied to feature f₆). Calculation of the exact relative contribution was not attempted. The features f₇ and f₈ are included to allow the ANN to determine the best blending of the features f₁ and f₆ versus the features f₇ and f₈ according to equations (2b) and (2c) as follows:

$$\begin{matrix}{f}_{7} = {f}_{1} / ( {f}_{1} + {ɛ}_{1} * {f}_{6} ) & ( 2 b ) \\ {f}_{8} = {f}_{6} / ( {f}_{1} + ɛ 2 * {f}_{6} ) & ( 2 c )\end{matrix}$$

where 0<ε₁<1 and 0<ε₂<1 and ε₁ does not have to be equal to ε₂.

FIG. 4D shows an image of a treatment field with corresponding features related to different ratios of areas. The impact on the radiation dose due to the interplay between the MLC and the Jaws (see FIG. 1B) is considered using a ratio of field opening area under the MLC and the Jaws as shown in equations (3a) and (3b) below, where the opening area of the MLC and the Jaws are denoted by A_{MLC} and A_{Jaw}, respectively, and the rectangular area defined by a maximum separation of an MLC pair in the radiation field is denoted by R_{MLC}.

$$\begin{matrix}{f}_{9} = {A}_{M L C} / {R}_{M L C} & ( 3 a ) \\ {f}_{10} = {A}_{M L C} / {A}_{Jaw} & ( 3 b )\end{matrix}$$

In order to keep the input range as [−1, 1], arbitrary scale factors were applied to the values for each of the features. The numerical values in FIGS. 4B and 4C are shown as examples for the radiation treatment field shown in FIG. 4A. The output of the ANN is also normalized by monitor unit (MU) and field opening area (i.e. feature f₁) and scaled to be in the range of [0, 1]. The input and output range scaling is desirable for an ANN that uses a sigmoidal function as a weighting factor for the various nodes.

The introduction of this feature extraction described above reduces the number of inputs significantly: i.e. from 160,000, which is the number of pixels for a 400×400 pixel fluence image, to the aforementioned 10 features. In some embodiments it may be possible to use a smaller number of features for the ANN, as the smaller number of features can still convey important information of the radiation treatment beam segment and allow efficient training of the ANN. Furthermore, it should be noted that in alternative embodiments, it may be possible to use other types of features especially with different radiation sensors and radiation sources. For example, other features may be used that include any arbitrary detector sensitivity and more detailed field shapes. In order to investigate the impact of the features on ANN performance, each group of features can be sequentially tested in a training process. Furthermore, while a small number of features with limited degrees of freedom (up to 2^{nd} order such as f₃ and f₅) was considered, improvement in performance may be possible by adding independent inputs that have a higher degree of freedom (e.g. 3^{rd} order or higher order) or adding features (f_{new}) that are a product of certain input features (e.g. f_{new}=fᵢ×fⱼ).

The number of hidden nodes directly impacts the degrees of freedom and generalization (i.e. reduction of overfitting) of the ANN at the same time. Different training methods such as, but not limited to, node pruning (which involves reducing a large number of initial hidden nodes and weights during training) or node expansion (which involves growing small networks during training) may be considered in order to find the optimal size of the ANN for more accurately predicting radiation measurements. Four different sets of hidden nodes (e.g. 3, 5, 10, and 20 nodes) for a single hidden layer were tested to investigate the best ANN configuration. Depending on the complexity of the modelling, multiple hidden layers may be required.

In the training and validation of the ANN, static and IMRT fields developed for RQCS commissioning and routine QA were used. In order to measure the effect of field size on the detector response, Area Output Factors (AOFs) with various shapes of rectangular fields ranging from 1 cm×1 cm to 40 cm×40 cm were programed into a single IMRT field consisting of multiple apertures. For the Elekta AOF field, a total of 95 apertures were used, while for the Varian AOF field, 66 apertures were used. For routine QA, small square fields of 4 cm×4 cm were irradiated at various locations on the RQCS detector. The number of QA fields was 62 and 48 for the Elekta and Varian radiation sources, respectively. Randomly selected clinical IMRT fields for treating the head-and-neck and prostate regions of various patients were also used for training and validation. The number of clinical IMRT segments was 483 and 318 for the Elekta and Varian radiation sources, respectively. Performance evaluation was performed on twelve MLP configurations with three different sizes of inputs (i.e. features) (e.g. 5, 8, and 10) and four different sizes of hidden nodes (e.g. 3, 5, 10, and 20). Ten untrained MLP children were evaluated for each configuration, totaling 120 MLPs since the MLP is a product of a stochastic process. Each MLP randomly selected 80% of the clinical field for training and the remaining 20% was used for performance evaluation. The results of all 10 MLPs in each group were combined for analysis. For training of the MLP, constant values of 0.1 and 0.5 were used for the momentum and learning rates, respectively. If larger values are used for the momentum and learning rates, then training can be done faster but there is an increased possibility of divergence (i.e. training failure). The values for the momentum and learning rates can be determined empirically. In general, a learning rate from 0.1 to 0.9 and a momentum rate from 0 to 0.5 are acceptable.

To determine the relative performance of the MLP, that is defined in accordance with the teachings herein, with a conventional analytical model approach, an MLP with 10 input features (as defined in FIGS. 24A-4B and equations 1-3) and 10 hidden nodes was compared with the RQCS analytic model IQM_Calc (Version number 310 which is described in a manual).

The uncertainty of the output of the RQCS analytical model depends on the accuracy of field definition and the machine output in terms of monitor unit (MU). The maximum error of the output of the RQCS analytical model is bounded by field size error (E_{FS}) and machine output error (E_{MU}) as shown in the equations (4a) to (4c):

$$\begin{matrix}{E}_{bound} = \sqrt{{E}_{FS}^{2} + {E}_{MU}^{2}} & ( 4 a ) \\ {E}_{F S} = \frac{{( F L + {Δ}_{M L C} )}^{2} - F {L}^{2}}{{FL}^{2}} \approx 2 \frac{{Δ}_{M L C}}{F L} & ( 4 b ) \\ {E}_{M U} = \frac{{Δ}_{M U}}{M U} & ( 4 c )\end{matrix}$$

where E_{bound} is the maximum error bound due to the field size error (E_{FS}) and machine output error (E_{MU}). These errors are modelled by effective square field size (FL²), MLCs or jaw positioning error (Δ_{MLC}), and the error in monitor unit (Δ_{MU}) at beam delivery. The minimum Δ_{MLC} and Δ_{MU} were found so that the error of the MLP calculation is less than the maximum error bound E_{bound} using the software routine fminsearch in Matlab™. Assuming there is a fixed amount of field size uncertainty (related to Δ_{MLC}) and a fixed amount of monitor unit uncertainty (Δ_{MU}), the error calculation may be inversely proportional to the field size and linearly proportional to the monitor unit.

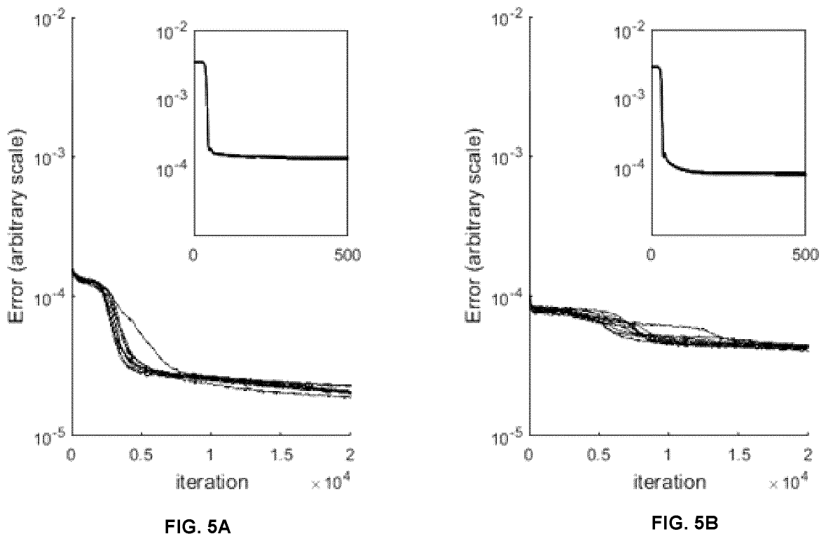

Referring now to FIGS. 5A and 5B, shown therein are plots showing the errors during MLP training for a Varian TrueBeam device and an Elekta Agility device. Matlab R2013b (MathWorks, Natick, Mass., US) was used to compute features from treatment fields and to simulate MLP training and validation on a personal computer. No hardware acceleration was performed since it only took about 1.5 sec to 1.7 sec to convert a DICOM file to fluence maps and to extract all 10 features from each IMRT segment and it only took about 370 sec and 270 sec, respectively, to train an MLP on Elekta and Varian data where the difference in computing time is mainly due to the number of fields (e.g. 640 vs. 432) used for training. The impact of the network size of the ANN was less than 20% (between using 3 hidden nodes and 20 nodes). The output calculation took less than 0.2 msec once the MLP was trained.

FIGS. 5A and 5B show a history of network training for MLPs with 10 features and 10 hidden nodes as an example. The initial error from an untrained MLP reduces by 100 fold in the first few hundred iterations of the training phase, and a few fold of further error reduction occurs in the following few thousand iterations. The training data set includes test static fields with a large variation in field size, shape, and off-axis distance in comparison to clinical fields. The training data also includes a random selection of 80% of available clinical IMRT fields. The remaining 20% of clinical IMRT fields were reserved for validation of MLP performance. The simulation was repeated 10 times with a different random selection of fields and a new MLP.

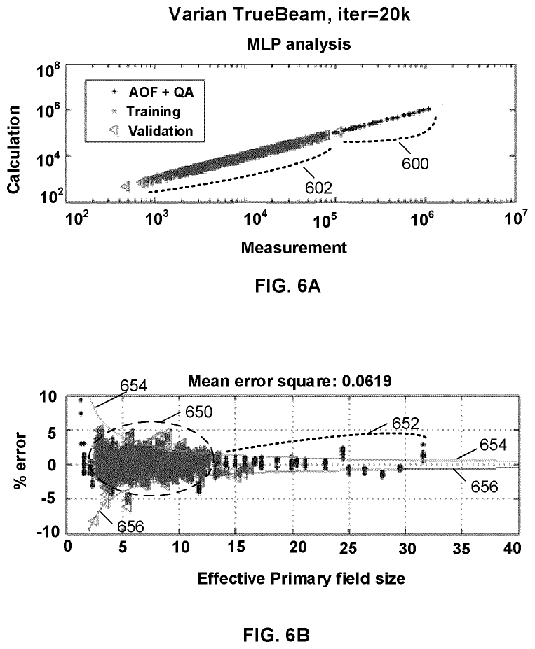

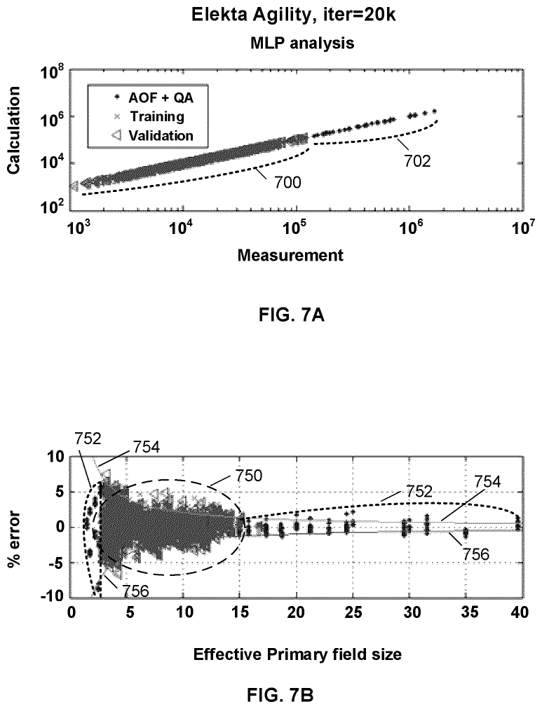

Referring now to FIGS. 6A-6B, shown therein are plots showing the correspondence between the calculations and predicted measurements during MLP training and the percentage error versus the effective primary field size during MLP training, respectively, for the Varian TrueBeam devices when using an MLP having the ten features f₁ to f₁₀ and 10 hidden nodes when using AOF and QA treatment fields used for commissioning and patient treatment. FIGS. 7A and 7B show the same measurements for the Elekta Agility device. In FIGS. 6A and 7A, the regions 600 and 700 correspond mainly to the validation and training data points while the regions 602 and 704 correspond to the AOF+QA data points. In FIGS. 6B and 7B, the regions 650 and 750 correspond mainly to the validation and training data points while the regions 652 and 754 correspond to the AOF+QA data points.

It can be seen that in FIGS. 6A and 6B there is a good one-to-one correspondence between the predicted radiation measurements and the actual radiation measurements. FIGS. 6B and 7B show the relative error as a function of field size and it can be seen that the smaller field size is sensitive to field size error. The relative error bound is shown in a solid line when a 1 mm field size error is assumed. The field size error here includes not only an error due to mechanical motion of the MLC/jaws but also an error in radio therapeutic modelling. In FIG. 6B, more than 97% of the fields for the TrueBeam device were within the error bound lines 654 and 656 when there is a 1 mm positioning error. In FIG. 7B, more than 95% of the fields of the Agility device were within the error bound lines 754 and 756 when there was a 1 mm positioning error.

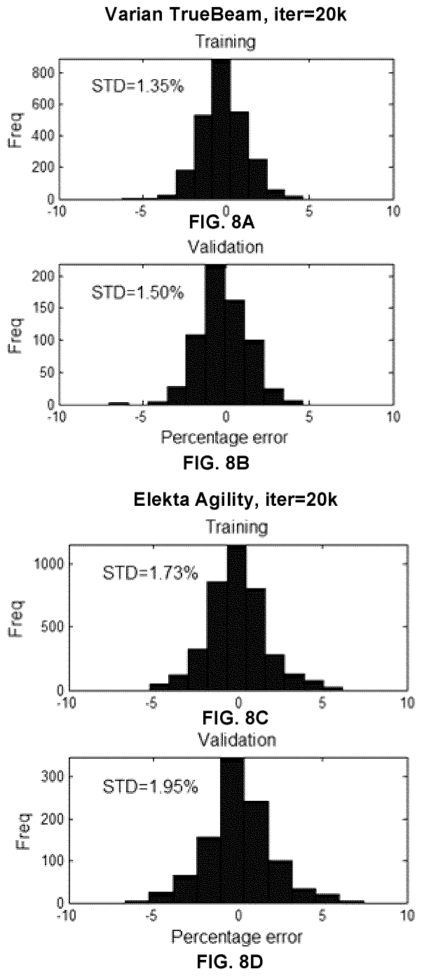

Referring now to FIGS. 8A-8D, FIGS. 8A-8B show histograms comparing the error distribution in training and validation on the Varian TrueBeam device while FIGS. 8C-8D show histograms comparing the error distribution in training and validation on the Elekta Agility device. These figures show that the relative error of the validation set is roughly similar to that of the test set. The standard deviation of error was 1.35% and 1.73% in training, and 1.50% and 1.95% in validation for the data from the Varian and Elekta devices, respectively. The Volumetric Arc Radiation Therapy (VMAT) field response was calculated using an MLP with 10 features f₁ to f₁₀ and 10 hidden nodes, trained with IMRT fields. Without further training for VMAT fields, the MLP successfully calculated the VMAT field from TrueBeam and Agility responses with high accuracy. The data demonstrates that the MLP is generalized and not sensitive to measurement noise.

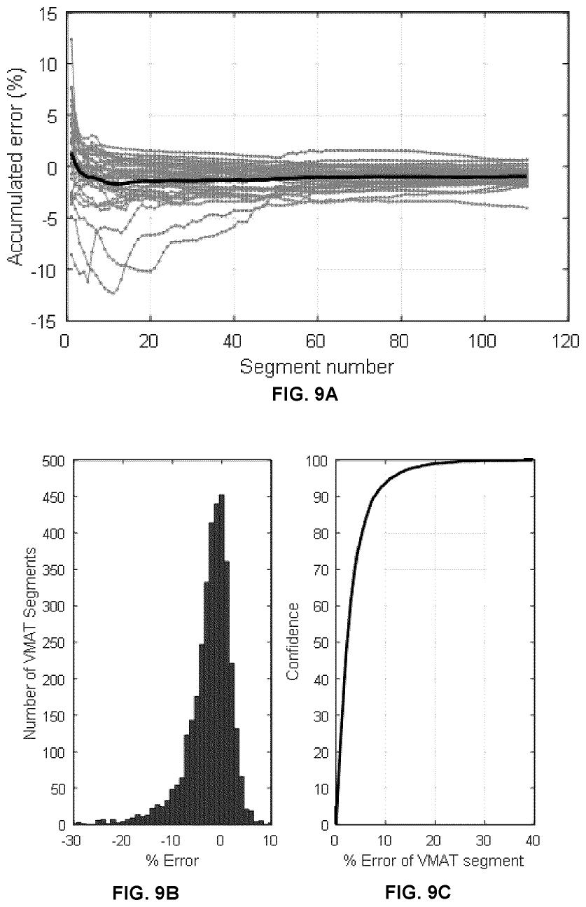

Referring now to FIGS. 9A-9C, shown therein are graphs showing the segment error for VMAT fields. FIG. 9A shows a plot of accumulated segment error for 32 VMAT fields. FIG. 9B is a histogram of VMAT field segments that shows similar performance to that of Intensity Modulated Radiation Therapy (IMRT) fields. FIG. 9C shows a plot of coverage versus percentage error derived from the area under the histogram of FIG. 9B. More than 95% of the accumulated segments show less than 3.6% error. The data was obtained with an MLP using the 10 features f₁ to f₁₀ and 10 hidden nodes that was trained with IMRT fields and not further trained with VMAT fields since training data such as QA and AOF fields are not available in VMAT mode and thus there is larger uncertainty that is inherent with VMAT fields.

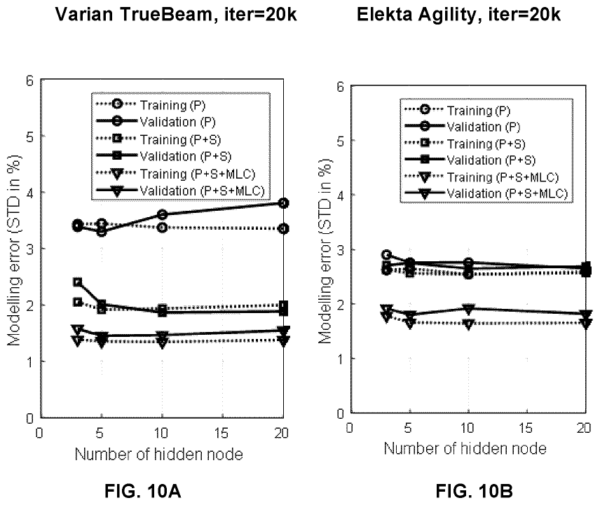

Referring now to FIGS. 10A and 10B, shown therein are plots showing modelling error based on the number of hidden nodes that are used in the MLP ANN for data obtained for the Varian TrueBeam and Elekta Agility devices, respectively. FIGS. 10A and 10B show the impact of input features and network size. It is more efficient to select a minimum number of features that convey as much information as possible on the physics of the RQCS measurement. The test results indicated that when using features based on primary radiation fluence, scatter (i.e. secondary) radiation fluence, and the effects of Jaw/MLC geometry on fluence using 10 features performed better than with fewer features for input to the MLP. The test results also indicate that MLPs with 5 to 10 hidden nodes perform similarly or better than MLPs with only 3 hidden nodes. However, MLPs with 20 hidden nodes show slightly worse error performance in training and much worse error performance in validation. This may be due to a loss of generalization or overfitting. Accordingly, MLPs with about 5 to 10 hidden nodes appeared adequate for the RQCS output prediction.

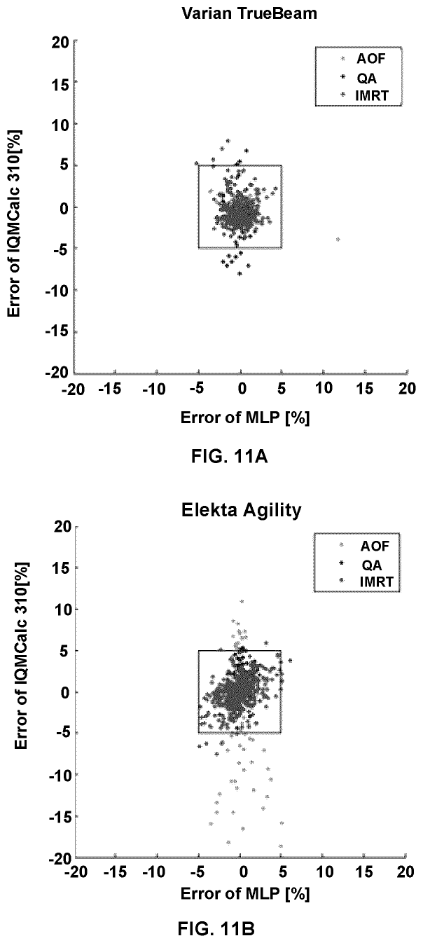

Referring now to FIGS. 11A and 11B, shown therein are plots comparing MLP error with the error for an analytic model for the Varian TrueBeam and Elekta Agility devices. The MLP ANNs performed better in general for all AOF, QA, and clinical IMRT fields as shown in FIGS. 11A and 11B. The analytic model, IQM_Calc, experienced difficulties, as shown in the error, for some test fields (e.g. for some of the area output factor and QA analytical measurements and measured detector responses), where an extremely small or large field size and a large off-axis beam were applied. Clinical IMRT fields were modelled reasonably well by both algorithms and showed good correlation between the two models for data from the Elekta Agility device.

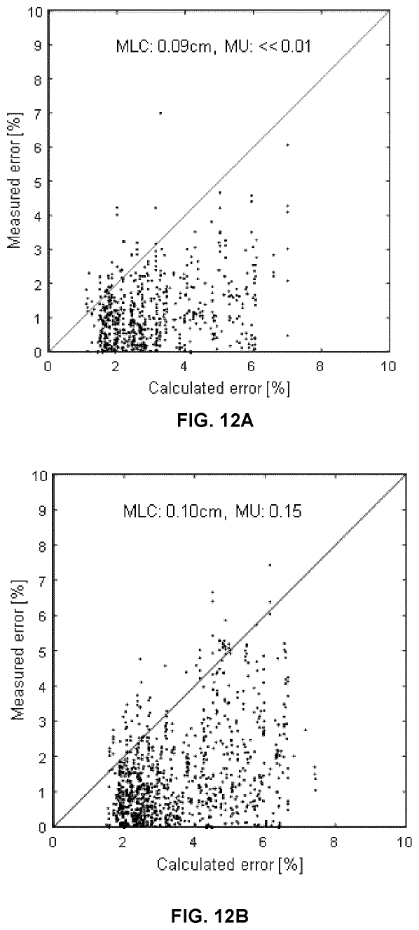

Referring now to FIGS. 12A and 12B, shown therein are plots comparing measured error with calculated error for different MLC and MU sizes. Modelling error as a function of field definition and MU uncertainty is shown. Field size uncertainty, Δ_{MLC}, and machine output uncertainty, Δ_{MU}, are estimated by 0.9 mm and less than 0.01 MU for the Varian TrueBeam device and 1.0 mm and 0.15 MU for the Elekta Agility device, respectively. This agrees well with observations from the log files of radiation treatment beam delivery. More than 93% of the measured errors were less than the maximum error bound, E_{bound}, calculated using equation (4) for both the TrueBeam and Agility analytical models. Since only two parameters were considered in error analysis, additional sources of uncertainty may be folded into the factors derived here, making them larger than determined independently.

Referring now to FIGS. 13A and 13B, shown therein are composite field fluences for QA and AOF fields. FIGS. 13A and 13B show a composite of the radiation source fields that were used as inputs during training of the ANN. These figures show that for both QA and AOF fields, the entire area of the radiation detector is covered as seen by the superposition of these test fields. The shapes and sizes of these fields are changed during commissioning and this approach was also taken for training the ANN.

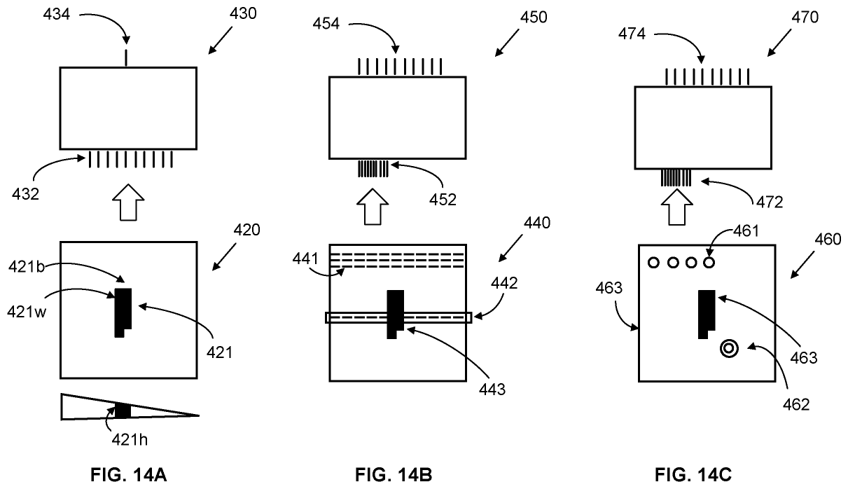

As described previously, the ANN can be applied to other radiation systems that employ different radiation sensors using a similar process that was developed for the RQCS radiation sensor discussed above. FIGS. 14A to 14C show schematic diagrams of example embodiments of different types of sensor configurations for radiation quality assurance (QA) systems. These sensors have different active areas for radiation detection. The sensors can be positioned at different locations such as at the entrance part of the radiation beam or the exit part of the radiation beam.

Referring now to FIG. 14A, shown therein is a schematic diagram of a radiation sensor 420 that can be used with the RQCS. The radiation sensor 420 is a single large-area sensor having an active region 421 that is defined by the jaws and the MLC. The active region generally has a height 421h, a width 421w and a length 421b. According to the teachings herein, the active region 421 of the radiation sensor 420 can be modeled using an ANN 430 that has an input having 10 features (f₁ to f₁₀ as described herein) and provides a single output 434. The ANN 430 can be an MLP or another suitable neural network.

Referring now to FIG. 14B, shown therein is a schematic diagram of a different radiation sensor 440 that has multiple linear arrays of line detectors 442 where each line detector comprises a plurality of small ion chambers 441. Each line detector 442 can be implemented so that it provides a line integral signal that corresponds to a unique one of the pair of leafs for the MLC when the sensor 440 is mounted at the collimator. For ease of illustration, only 4 line detectors are shown and only one ion chamber is labeled. Each given line detector 442 generates a measurement signal that is a composite of the radiation measured around each radiation detector 441 in the given line detector 442. It can be assumed that there are Y line detectors that may be uniformly spaced to cover a majority of the sensor 440. Since the activation (i.e. radiation beam) pattern 443 depends on the geometry of the jaws and the MLC, the same 10 features can be used as in the case of the large area ion detector 420 but they can be applied to each linear detector 442. It should be noted that the large area ion detector 420 may or may not have a gradient. For example, a non-gradient ion chamber can be used for a very small field (located at the center of collimator) to monitor Stereotactic Radiosurgery. Therefore, according to the teachings herein, the radiation sensor 440 can be modeled using an ANN 450 that has an input 452 having a linear array of values (i.e. Y*10 input features (f₁ to f₁₀ as described herein)) and provides an output 454 having a linear array of values, with one value being associated with each line detector 441. In this case, the ANN 430 can be scaled to create the ANN 450. The ANN 450 is then trained in a similar manner as ANN 430 (simply there are more inputs and outputs) to accurately predict the radiation response signal measurements from each linear array of line detectors. In an alternative embodiment, Y ANNs can be trained where each ANN corresponds to each line detector 441. The ANN 470 can be implemented in a same manner with simply more inputs and outputs using an MLP or another suitable neural network. For example, in an alternative embodiment, a convolutional neural network may be used as the ANN 450 since it is good for modelling narrow or restricted detection regions in which the detection regions from each detector do not overlap with one another in contrast with a large area ion detector such as with radiation sensor 440. A convolutional neural network is suitable in these situations because the nodes are not all connected meaning that the nodes at a given stage are not all connected to the nodes in a previous stage or the nodes in a subsequent stage. For example, each input node is not connected to each node in a hidden layer and each node in a hidden layer is not connected to each output node.

Referring now to FIG. 14C, shown therein is a schematic diagram of another radiation sensor 460 with a plurality, for example hundreds, of point-sensor detectors 461. An example of the active area 462 of a given point detector is shown. Each detector 461 provides an output. The outputs of the detectors 461 for are combined provide an output. It can be assumed that there are Y×N detectors 461 that may be uniformly spaced to cover a majority of the sensor 460 in a 2D matrix where Y is an integer indicating the number of rows and N is an integer indicating the number of columns of the 2D matrix, where Y and N are greater than zero. Accordingly, the overall activation pattern 463 may be similar to the radiation sensor 420 of FIG. 14A as it depends on the geometry of the jaws and the MLC. As a result, the same 10 features can be used as in the case of the large area ion detector 420 but they can be applied to each point-sensor detector 461. Therefore, according to the teachings herein, the radiation sensor 460 can be modeled using an ANN 470 that has an input 472 having an array of values (i.e. Y*X*10 input features (f₁ to f₁₀ as described herein)) and provides an output 474 having a 2D matrix array of values where the array has a size of Y*X, with one value being associated with each point detector 461. In this case, the ANN 430 can be scaled to create the ANN 470. The ANN 470 is then trained in a similar manner as the ANN 430 (except there are many more inputs and outputs) to accurately predict the radiation response signal measurements from a 2D array of point detectors. In an alternative embodiment, Y*X separate ANNs can be trained with one ANN for each radiation detector. With this approach the total number of weights should be smaller. The ANN 470 can be implemented using an MLP or another suitable neural network. For example, in an alternative embodiment, a convolutional neural network may be used as the ANN 470 for the same reasons given above for radiation sensor 440.

In another alternative embodiment in which the radiation sensor comprises a 3D arrangement of radiation detectors in a cylindrical, cubical or other geometrical format. The 3D arrangement generally includes N groups of Z radiation detectors resulting in a total of N*Z radiation detectors where N and Z are integers that are greater than zero. In this case a single ANN can still be used in which there are N*Z*F inputs and N*Z outputs, where F is an integer representing the number of input features that are used where F is greater than zero. Alternatively, there can be N ANNs which each has Z*F inputs and Z outputs. Each of these ANNs may be implemented using an MLP, a convolutional neural network or another suitable neural network.

For each of the radiation sensors 420, 440 and 460, the following derivation is provided to show how the input features can be defined based on the response signal provided by the radiation detector. In general, for each of these detectors, a detector signal, dS, contributed from a small sub area, dA, can be described by equation (5):

$$d S = {ΨΥ}_{d} d A \qquad ( 5 )$$

where Ψ the energy fluence of a radiation beam and Υ_{d} is a relative response of the detector in the corresponding sub area, dA. The total radiation response signal S can be found by collecting signals from all active sub areas of the detector. Energy fluence from a linear accelerator is often modeled by two radiation sources—one at the Tungsten target for the primary beam and the other from a flattening filter. The total radiation response signal S can be described by equations (6a) and (6b):

$$\begin{matrix}S = \int {ΨΥ}_{d} dA & ( 6 a ) \\ S = \int ( {Ψ}_{p} + {Ψ}_{s} ) {Υ}_{d} dA & ( 6 b )\end{matrix}$$

In the radiation sensor 420, the detector response, Υ_{d}, is a function of sensitivity direction. The sensitivity direction can be denoted by x, but it can be any arbitrary direction depending on the application, including ±x or ±y. Since energy fluence in a LINAC is a function of the distance from the center of radiation isocenter, r, the detector signal can be described by equation (7a) or equation (7b).

$$\begin{matrix}S = \int ( \overline{{Ψ}_{p}} ( {p}_{0} + {p}_{1} r + {p}_{2} {r}^{2} + … ) + \overline{{Ψ}_{s}} ( {s}_{0} + {s}_{1} r + {s}_{2} {r}^{2} + … ) ) ( 1 + {d}_{1} x + {d}_{2} {x}^{2} + … ) dA & ( 7 a ) \\ S = {p}_{0} \int \overline{{Ψ}_{p}} dA + {d}_{1} \int \overline{{Ψ}_{p}} xdA + {d}_{2} \int \overline{{Ψ}_{p}} {x}^{2} dA + … + {p}_{1} \int \overline{{Ψ}_{p}} rdA + {p}_{2} \int \overline{{Ψ}_{p}} {r}^{2} dA + … + {s}_{0} \int \overline{{Ψ}_{s}} dA + … & ( 7 b )\end{matrix}$$

Features 1 to 6 of the ANN 430 are found in the above equations. In order to consider the relative contribution of the primary and scatter radiation sources, features 7 and 8 are adapted. For field shape consideration, features 9 and 10 are applied. Since the area integral is on the active area (represented by dA in the equations (5) to (7b)) for the given detector, it is the whole detector area for the radiation detector 420. For the radiation detector 440, the active area of each electrode is limited to the corresponding area of a leaf pair and is a rectangular area around each line detector. For the radiation detector 460, the active area of each discrete detector is limited to the small area around the center of the detector.

It should be noted that in alternative embodiments, the radiation sensor may comprise two of the radiation sensors 420 in a “double-stacked configuration” such that the two linear gradients are parallel and opposing to one another or are orthogonal to one another to provide spatial sensitivity in the entire detecting area. In this case there are two radiation detector outputs and there are two ANNs that each have a set of input features f₁ to f₁₀ and each provide an output for a total of two outputs. In yet another alternative embodiment, the radiation sensor may comprise four of the radiation sensors 420 in a “quadruple stacked configuration” which are essentially two double stacked large area ion sensors. In this case there are four radiation detector outputs and this radiation sensor can be modelled using 4 ANNs that each have a set of input features f₁ to f₁₀ and each provide an output for a total of four outputs.

In at least one alternative embodiment, the input features that are used by an ANN include the radiation treatment field parameters described above (i.e. features f₁ to f₁₀) as well as additional features. For example, the additional features include, but are not limited to, at least one of, radiation source model, MLC model, beam energy, type of radiation sensor, and radiation sensor location. For example, the radiation sensor location can indicate whether the radiation sensor is in a direct path of the beam before the patient (i.e. entrance beam monitoring) or after the patient (i.e. exit beam monitoring), or in an indirect path such as any scattered radiation path (i.e. the radiation detector is placed anywhere outside of the radiation path such as, but not limited to, on the wall, for example, so that the radiation detector does not intercept the primary radiation beam but rather the radiation beam may reflect off an object such as the patient/couch and then intersect the radiation sensor). Furthermore, the type of radiation sensor can be a large area ion detector, a large area gradient ion detector, at least two large area gradient ion detectors in an inverse parallel configuration or an orthogonal configuration, a series of line detectors, a 2D array of point detectors or a 3D array of point detectors.

In another alternative embodiment, the ANN is used in combination with an analytic radiation measurement method (e.g. IQM_Calc) in a hybrid mode where the ANN is trained and configured to generate predicted analytical errors which is the difference between the measured radiation dose and the analytical radiation measurement generated by the analytic radiation measurement method. In this case, the predicted analytical radiation measurement is also an provided as an input in addition to the input features that were previously described as being provided to the ANN. This mode of use can provide results which can be more interpretable since there may be situations in which an AI/ANN based radiation measurement may be incorrect for an unusual situation, for which the AI/ANN is not sufficiently trained. However, with a hybrid system, the “first order” results may be obtained using an analytical method, and the ANN may be used to help fine tune the final measurement results.

It should be understood that when training any of the ANNs described herein that in addition to using radiation treatment parameters for a variety of QA and AOF fields, the training data can also include data that was obtained from various types of radiation source manufacturers, different radiation source models (e.g. different collimator types), different amounts of beam energy, and different beam calibration units. For example, a user may determine that at one cancer treatment center, 1 MU (Monitor Unit) of radiation released by the LINAC that is used may provide 1 cGy of radiation dose at a distance of 100 cm from the radiation source (at a depth of 1.5 cm of water) for a 10 cm×10 cm field. However, for another cancer center that uses a different LINAC, 1 MU of radiation released by the LINAC may provide 1 cGy of radiation dose at a distance of 101.5 cm distance from the radiation source for the same field size and depth in water. Therefore, both of these LINACs will require different calibration.

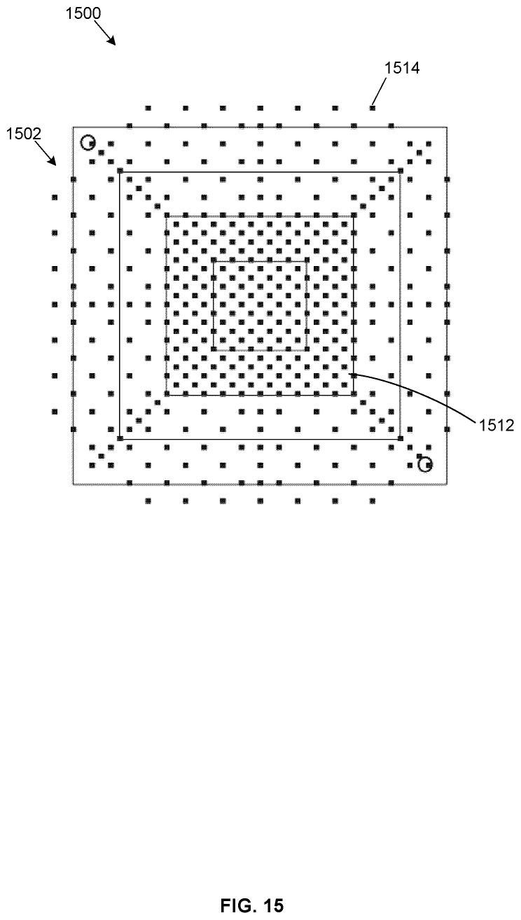

Referring now to FIG. 15, shown therein is a schematic diagram of an example embodiment of a radiation sensor 1500 that comprises a two-dimensional (2D) detector array 1502 that may be used with the radiation dose monitoring system 10 of FIG. 1A. The 2D detector array 1502 comprises 445 diode detectors mounted on a flat panel, with an effective build-up depth of 5 cm of solid water. Each dot represents a diode detector of size 0.8 mm×0.8 mm. The spacing for the inner diodes 1512 is 5 mm, and the spacing for the outer diodes 1514 (outside of the 10 cm×10 cm region) is 10 mm. The radiation sensor 1500 was implemented using the MapCheck radiation sensor, which is manufactured by Sun Nuclear.

A study was conducted in which the MapCheck radiation sensor was positioned on the treatment couch, at a distance of 100 cm from the radiation source 12. However, the study simulates the operation of a 2D detector array mounted at the collimator of the radiation source. The results of the study should be indicative of representing any 2D detector array mounted at the collimator with an ANN. The radiation sensor 1500 was exposed to IMRT beams (a set of beams with rectangular apertures and typical treatment beams), and data was collected with the radiation source 12 (i.e. the LINAC) in a reference working condition. For initial ANN training, a total of 157 beam segments with varying field sizes and off-axis locations were used to characterize the pair of the LINAC and the radiation sensor 1500. Each ANN consisted of 9 nodes in the input layer, corresponding to 9 features derived for each beam segment, and one hidden layer with 10 nodes, and one output node corresponding to each of the diodes. There is one ANN for each diode in the 2D detector array 1502.

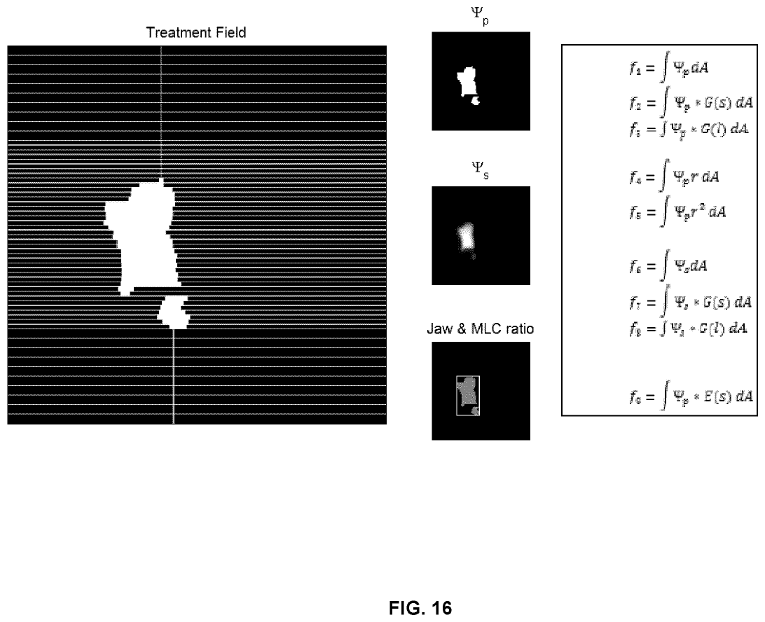

Referring now to FIG. 16, shown therein is an example of an image of an example treatment field, beside which are example images of certain example features of radiation field segments. While images shown in FIG. 16 as the same as those shown in FIGS. 4A-4B, the features that can be used as inputs to the ANNs are different as shown in equations 8a to 10a.

$$\begin{matrix}{f}_{1} = \int {Ψ}_{p} dA & ( 8 a ) \\ {f}_{2} = \int {Ψ}_{p} * G ( s ) dA & ( 8 b ) \\ {f}_{3} = \int {Ψ}_{p} * G ( l ) dA & ( 8 c ) \\ {f}_{4} = \int {Ψ}_{p} r dA & ( 8 d ) \\ {f}_{5} = \int {Ψ}_{p} {r}^{2} dA & ( 8 e ) \\ {f}_{6} = \int {Ψ}_{s} dA & ( 9 a ) \\ {f}_{7} = \int {Ψ}_{s} * G ( s ) dA & ( 9 b ) \\ {f}_{8} = \int {Ψ}_{s} * G ( l ) dA & ( 9 c ) \\ {f}_{9} = \int {Ψ}_{p} * E ( s ) dA & ( 10 a )\end{matrix}$$

Five features were extracted from the primary fluence including the first feature (f₁) which is the primary fluence, the second and third features (f₂ and f₃) which are low pass filtered versions of the primary fluence where the filtering uses small and large Gaussian kernels, G(s) and G(l), and considers photon scatter in solid water phantom placed in front of detector and the fourth and fifth features (f₄ and f₅) which account for photon beam flatness for the particular LINAC that was used in this study. The small and large Gaussian kernels are a superposition of two Gaussian functions to account for realistic spread of a radiation beam. For example, a Gaussian filter with a small kernel may be applied to areas that are about 5 mm in radius while a Gaussian filter with a larger kernel may be applied to areas that are 30 mm in radius. In equations 8d and 8e the variable r represents the radial distance from the radiation detector center. It should be noted that while the features f₂ and f₃ for the large area gradient ion radiation sensor takes into account the detector's “linear” gradient sensitivity, here the features f₂ and f₃ take into account the (point) detector sensitivity in terms of 2 Gaussian functions for the radiation sensor 1500. The sixth, seventh and eighth features (f₆, f₇, and f₈) account for the extended source contribution. The ninth feature (f₉) is used to account for the field edge effect that is defined by Jaw and MLC using an edge detection filter. In particular, it should be noted that while the feature f₉ for the large area gradient ion radiation sensor accounts for integrating the edges of the radiation beam segments into one signal, here the feature f₉ accounts for the edges of the radiation beam segments to provide individual signals for each detector of the radiation sensor 1500. In equation 10a, the function E(s) is an edge detection filter and optionally a small kernel size may be used for the edge detection filter. All area integration is performed around each individual detector. Because of the fact that there is no varying sensitivity in this type of radiation sensor and an extra phantom is used, the features f₂ and f₃ are modified from those used for modelling the RQCS large area gradient ion radiation sensor using an ANN.

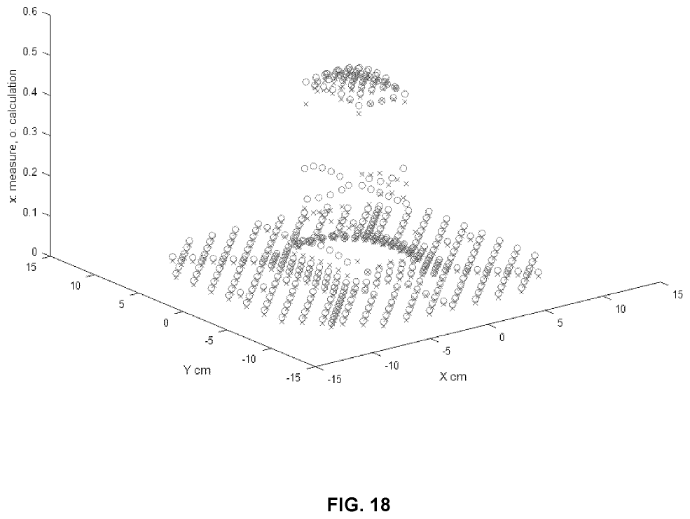

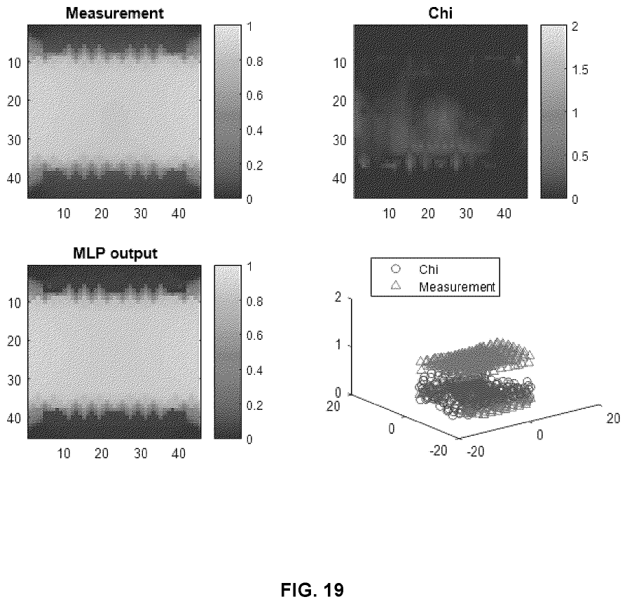

About 5% of the data was randomly selected for training the MLPs from a total of 92,560 collected data. A trained set of MLPs (i.e. one MLP for each detector in the 2D detector array) was developed and tested on the fields from prostate and head & neck IMRT plans irradiated on the Elekta Infinity system. Training took about 30 minutes on a desktop computer (i.e. Intel i5-6500 CPU with 16 GB of RAM) using Matlab code. To assess the validation of the trained network, the output of the MLP for each of the detectors was compared with the corresponding measured signal. Most discrepancy was found at the edges of the field segments. The modelling error within the radiation field, neglecting the edge and penumbra, was found to be 0.24%±2.45% (mean±standard variation), as shown in FIGS. 17 and 18. In particular, FIG. 17 shows a histogram showing the difference (% error) between the measured and ANN predicted signals. FIG. 19 shows a 3D plot showing the measured and ANN predicted signals, with the results showing some large errors at the edges of the field, which is due mainly to the beam penumbra.

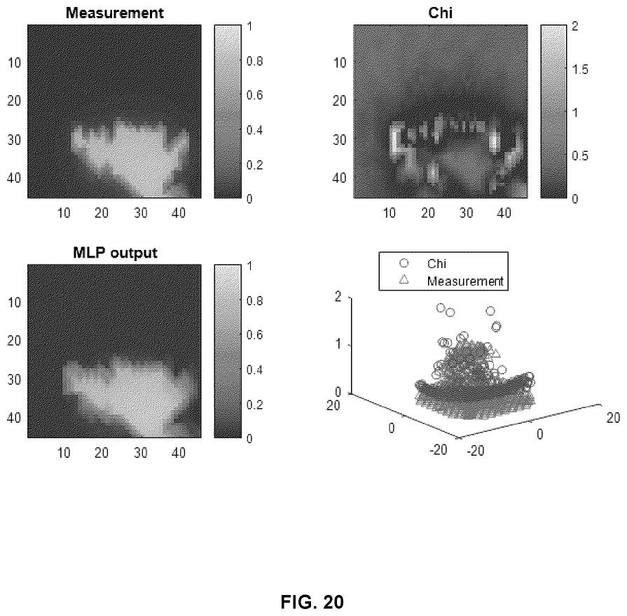

Simplified Gamma analysis (x) showed good agreement between the measured radiation dose and the radiation dose prediction by the ANN model. In particular, the analysis showed >97.3% pass rate for the criteria of 3 mm (distance to agreement); 3% difference in signal and a threshold of 3%. A graphical representation of the Gamma analysis is shown in FIGS. 19 and 20. FIG. 19 shows the measured radiation dose and the ANN predicted measurements of a large field (i.e. rectangular 40 cm×25 cm), where the Gamma (Chi) values are less than 1, indicating a good (passing) agreement. FIG. 20 shows the measured radiation dose and the ANN predicted measurements of a clinical IMRT field segment (i.e. an irregularly shaped smaller field to conform to the target), where the Gamma (Chi) values are mostly less than 1. However, the values corresponding to the edges of the field apertures are shown to have failed (more than 1), which is due to a combination of small uncertainties in radiation beam delivery, as well as due to positioning uncertainty of the 2D detector array 1502.

As a proof of principle, this study suggests that the approach outlined above can effectively model a radiation sensor comprising a 2D detector array for radiotherapy beam monitoring. The ANN can be utilized to predict signals of the radiation dose monitoring system, when used either for a pre-treatment QA of treatment beams, or during treatment delivery, in which case the radiation sensor is mounted at the collimator, as was shown and explained in FIG. 1B. The principle applied here for 2D detector array can be extended to a 3D detector array, in which case the ANN output may be used for training to improve accuracy as well as to generate the predict measurement signals for each of the detectors in the array. The extension involves accounting for the detector sensitivity. For example, for a large area ion gradient sensor an approximate linear gradient is used and for a 2D array of detectors, each detector sensitivity may be represented by 2D Gaussian type functions. Accordingly, for a radiation sensor with a 3D matrix of detectors, each detector may have a sensitivity that can be represented by a 3D Gaussian.

The embodiments of the present disclosure described above are intended to be examples only and it is not intended that the applicant's teachings be limited to such embodiments. The present disclosure may be embodied in other specific forms. Alterations, modifications, and variations to the disclosure may be made without departing from the intended scope of the present disclosure. While the systems, devices, and processes disclosed and shown herein may comprise a specific number of elements/components, the systems, devices, and assemblies may be modified to include additional or fewer of such elements/components. For example, while any of the elements/components disclosed may be referenced as being singular, the embodiments disclosed herein may be modified to include a plurality of such elements/components. Selected features from one or more of the example embodiments described herein in accordance with the teachings herein may be combined to create alternative embodiments that are not explicitly described. All values and sub-ranges within disclosed ranges are also disclosed. The subject matter described herein intends to cover and embrace all suitable changes in technology.

### REFERENCES

1. Amiri S, Movahedi M M, Kazemi K, and Parsaei H, “An Automated MR Image Segmentation System Using Multi-layer Perceptron Neural Network”, J. Biomed. Phys. Eng. 2013; 3(4), pp. 115-122.
2. Boyer A and Yu C, “Intensity-modulated radiation therapy with dynamic multileaf collimators”, Semin. Radia. Oncol., 1999, 9 (1), pp. 48-59.
3. Egmont-Petersen M, de Ridder D D, and Handels H, “Image processing with neural networks-a review”, Pattern Recognition, 2002, 35: pp. 2279-2301.
4. Hetcht-Nielsen R, “Kolmogorov mapping neural network existence theorem”, IEEE Int. Conf. Neural Networks 2, 1989, pp. 359-366.
5. Hinton G and Salakhutdinov R, “Reducing the Dimensionality of Data with Neural Networks”, Science, 2006, 313: pp. 504-507.
6. Hoffman D, Chung E, Hess C, Stern R and Benedict S, “Characterization and evaluation of an integrated quality monitoring system for online quality assurance of external beam radiation therapy”, J. Appl. Clin. Med. Phys., 18: pp. 40-48.
7. Hornik K, Stinchcombe M and White H, “Multilayer feedforward networks are universal approximators”, Neural Networks 2, 1989, pp. 359-366.
8. Islam M K, Norrlinger B D, Smale J R, et al., “An integral quality monitoring system for real-time verification of intensity modulated radiation therapy”, Med. Phys. 2009, 36: pp. 5420-5428.
9. Irie B and Miyake S, “Capabilities of three-layered perceptions”, IEEE Int. Conf. Neural Networks 1, 1988, pp. 641-648.
10. Jaffray D and Siewerdsen J, “Cone-beam computed tomography with a flat-panel imager: Initial performance characterization”, Med. Phys., 2000, 27(6), pp. 1311-1323.
11. Kallenberg M, Petersen K, Mielsen M, et. al. “Unsupervised deep learning applied to breast density segmentation and mammographic risk scoring”, IEEE Trans. Med. Imaging, 2016, vol. 35, 5, pp. 1322-1331.
12. Raaijmakers A J, Hardemark B, Raaymakers B W, Raaij-makers C P, and Lagendijk J J., “Dose optimization for the MRI-accelerator: IMRT in the presence of a magnetic field”, Phys. Med. Biol., 2007, 52: pp. 7045-7054.
13. Sifaoui A, Abdelkrim A, and Benrejeb M, “On the Use of Neural Network as a Universal Approximator”, International Journal of Sciences and Techniques of Automatic control & computer engineering, 2008, 2, pp. 386-399.
14. Webb S, “Optimizing the planning of intensity modulated radiotherapy”, Phys. Med. Biol., 1994, 39 (12), pp. 2229-2246.

1. A radiation dose monitoring system for monitoring an amount of radiation in a radiation beam generated by a radiation source for a radiation treatment session, wherein the system comprises:
a radiation sensor that is positioned in a path of the radiation beam and is configured to provide an actual radiation measurement of an amount of radiation in the radiation beam;
an interface unit, operatively coupled to the at least one radiation sensor;
a memory unit; and
a processor, operatively coupled to the interface unit and the memory unit, the processor being configured to:
obtain radiation treatment plan data for the radiation treatment session;
extract a plurality of feature values for features of radiation field segments from the radiation treatment plan data for the radiation treatment session;
generate a predicted radiation measurement using an artificial neural network engine that receives the plurality of feature values as inputs; and
determine an error measurement between the actual radiation measurement and the predicted radiation measurement.

2. (canceled)

3. The system of claim 1, wherein the processor is further configured to send a notification output signal to an operator of the radiation source when the error measurement is outside a predetermined safe operation range for the amount of radiation defined in the radiation treatment plan data.

4. The system of claim 1, wherein the processor is further configured to generate a control signal that is provided to the radiation source to:
stop the generation of the radiation beam when the error measurement is outside of a predetermined safe operating range for the amount of radiation defined in the radiation treatment plan data or
adjust the amount of radiation in the radiation beam that is generated by the radiation source when the error measurement is outside of a predetermined safe operating range for the amount of radiation defined in the radiation treatment plan data.

5. (canceled)

6. The system of claim 1, wherein the features of the radiation field segments comprise spatial variation of energy fluence, positional sensitivity of the radiation sensor, contribution of a secondary radiation source and shape of field opening area.

7. The system of claim 1, wherein the radiation sensor comprises: (a) a large area gradient ion chamber or (b) the radiation sensor comprises two large area gradient ion chambers in a stacked configuration having parallel and opposing gradients or having orthogonal gradients, each ion chamber being adapted to provide an output vale for the actual radiation measurement, and the ANN engine is configured to use 10 features of the radiation field segments as input features.

8. (canceled)

9. The system of claim 7, wherein the features for the variation of energy fluence include: ƒ₄=∫Ψₚr dA and ƒ₅=∫Ψₚr²dA where Ψₚ is energy fluence due to a primary radiation source, r is a radial distance from a center of a treatment beam area defined by jaw and Multileaf Collimator geometry of the radiation source and the integral is taken over the treatment beam area.

10. The system of claim 7, wherein the features for the positional sensitivity of the radiation sensor include: ƒ₁=∫ΨₚdA, ƒ₂=∫ΨₚxdA and ƒ₃=∫Ψₚx²dA where Ψₚ is energy fluence due to a primary radiation source, x is a direction of a Multileaf Collimator or a direction of detector sensitivity and the integral is taken over the treatment beam area defined by jaw and Multileaf Collimator (MLC) geometry of the radiation source.

11. The system of claim 7, wherein the feature of contribution of a secondary radiation source include ƒ₆=∫ΨₛdA where Ψₛ is energy fluence due to a secondary radiation source, and the integral is taken over the treatment beam area defined by jaw and Multileaf Collimator geometry of the radiation source.

12. The system of claim 10, wherein the feature of contribution of shape of field opening area include f₇=f₁/(f₁+ε₁*f₆) and f₈=f₆/(f₁+ε₂*f₆) where 0<ε₁<1 and 0<ε₂<1.

13. The system of claim 7, wherein the features of the shape of field opening area include ƒ₉=A_{MLC}/R_{MLC} and ƒ₁₀=A_{MLC}/A_{Jaw} where A_{MLC} and A_{Jaw} are opening areas of an MLC and Jaws of the radiation source, respectively, and R_{MLC} is a rectangular area defined by a maximum separation of an MLC pair in the radiation field.

14. The system of claim 1, wherein the radiation sensor comprises a plurality of point detectors in a two dimensional array with Y rows and N columns where each point detector provides an output value for the actual radiation measurement and the ANN engine employs an ANN for each of the point detector or a single ANN with F*Y*N inputs to generate a two dimensional array of output values for the predicted radiation measurement, where F is a number of input features and F, Y and N are integers greater than zero.

15. The system of claim 14, wherein the features for the variation of energy fluence include: ƒ₄=∫ΨₚrdA and ƒ₅=∫Ψₚr²dA where Ψₚ is energy fluence due to a primary radiation source, r is a radial distance from a radiation detector center and the integral is taken over an area around each of the point detectors; and
wherein the features of the primary fluence measured by the radiation sensor include: ƒ₁=∫ΨₚdA, ƒ₂=∫Ψₚ*G(s)dA and ƒ₃=∫Ψₚ*G(l)dA where Ψₚ is energy fluence due to a primary radiation source, and G(s) and G(l) are small and large Gaussian kernels and the integral is taken over an area around each of the point detectors.

16. (canceled)

17. The system of claim 14, wherein the feature of contribution of a secondary radiation source include ƒ₆=∫ΨₛdA, ƒ₇=∫Ψₛ*G(s)dA and ƒ₈=∫Ψₛ*G(l)dA, where Ψₛ is energy fluence due to a secondary radiation source, G(s) and G(l) are small and large Gaussian kernels and the integral is taken over an area around each of the point detectors; and
wherein the feature for accounting for edges of the radiation beam segments includes ƒ₉=∫Ψₚ*E(s)dA where E(s) is an edge filter and the integral is taken over an area around each of the point detectors.

18. (canceled)

19. The system of claim 1, wherein the radiation sensor comprises Y line detectors that each provide an output value for the actual radiation measurement and the ANN engine employs an ANN for each line detector or a single ANN with F*Y inputs to generate a linear array of output values for the predicted radiation measurement, where F is a number of input features and F and Y are integers greater than zero.

20. The system of claim 1, wherein the radiation sensor comprises a 3D arrangement of radiation detectors, where the 3D arrangement includes N groups of Z radiation detectors and the ANN engine employs an ANN for each group or a single ANN with N*Z*F inputs and N*Z outputs, where F is an integer representing the number of input features that are used where F, N and Z are integers that are greater than zero.

21. The system of claim 1, wherein the ANN engine is configured to use additional input features including at least one of radiation source model, MLC model, beam energy, type of radiation sensor, and radiation sensor location.

22. The system of claim 1, wherein the ANN engine is configured to use additional input features comprising patient geometry at a treatment region, location of the patient on a treatment table and radiation sensor location including immediately positioned before the patient for entrance beam monitoring or positioned after the patient for exit beam monitoring.

23. The system of claim 1, wherein the ANN engine is configured to use a multi-layer perceptron (MLP) neural network or a convolutional neural network.

24.-27. (canceled)

28. The system of claim 1, wherein the ANN engine is configured to use N ANNs to generate N intermediate predicted radiation measurements that are statistically combined to provide the predicted radiation measurement, where N is an integer greater than one.

29.-30. (canceled)

31. A method for monitoring an amount of radiation in a radiation beam generated by a radiation source for a radiation treatment session, wherein the method comprises:
obtaining an actual radiation measurement of an amount of radiation in the radiation beam from a radiation sensor that is positioned in a path of the radiation beam; and
at a processor:
extracting a plurality of feature values for features of radiation field segments from the radiation treatment plan data for the radiation treatment session;
generating a predicted radiation measurement using an artificial neural network engine that receives the plurality of feature values as inputs; and
determining an error measurement between the actual radiation measurement and the predicted radiation measurement.

32.-60. (canceled)
